SEQUENCE LISTING (72 sequences)

SEQ ID NO: 1 - protein, 388 aa, Arabidopsis thaliana
MALLRPHLHRFHSNTLRHSAYPSADAGGGLVVYPTYGRHRCSAIAIDAPSSLTGVTPIRWGYTSVQGFRDEMEDDIVIRSDAVDSFSYAAVFDGHAGSSSVKFLREELYKECVGALQAGSLLNGGDFAAIKEALIKAFESVDRNLLKWLEANGDEEDESGSTATVMIIRNDVSFIAHIGDSCAVLSRSGQIEELTDYHRPYGSSRAAIQEVKRVKEAGGWIVNGRICGDIAVSRAFGDIRFKTKKNDMLKKGVDEGRWSEKFVSRIEFKGDMVVATPDIFQVPLTSDVEFIILASDGLWDYMKSSDVVSYVRDQLRKHGNVQLACESLAQVALDRRSQDNISIIIADLGRTEWKNLPAQRQNVVVELVQAATTIGLVTVGIWMSSHLS

SEQ ID NO: 2 - protein, 386 aa, Vitis vinifera
MALLSQQLQRFFLTTLNCTSNLKATKKNNSVALKERARCCSAIAIDAPSFSGVAGIRWGLCRLQGPREEMEDEAVVRSDGLDGFSFAAVFDGHAGFSSVKFLRDELYKDCVAALQGGLLLSGKNFNIIREALEKAFESADAKLLNWLETTGEDVESGSTATVLLIGDDMVFISHVGDSCVVLSRSGKAEELTNPHRPYGSNKSSLEEIRRIREAGGWIVNGRICGDIAVSRSFGDMRFKTKKNEMLEKGLEEGRWSQKFVSRVQFTGDLVVASPDVFQVALGSDAEFLLLASDGLWDYMNSSEAVTFVRNELRQHGDVQVASEALARAALDRRTQDNVSIIIADLGRTDWQSLPLQQQNLFLELVQALGTIGIVTLGIWMSSQFSL

SEQ ID NO: 3 - protein, 389 aa, Oryza sativa
MALLSPRVPRLPLASASAAGAGLRCCVSGGRAGSAAWCHASAAGSVASSSSELEAIRWGTAKLQGARDEMEDEVVLRPGSLLDGFSFAAVFDGHAGFSAVEFLRDELYKECAAALDGGAVLSTKNLDAITDSIQRAFATVDANLSTWLEQMDKEDESGATATAMFLRNDVLVVSHIGDSCLQVVSRGGRPQAVTNFHRPYGNKKASLEEVKRIRAAGGWIVDGRICGEISVSRAFGDIRFKTRKNEMLVKGVKEGRWTEKFISRINFKGDLIVSSPDVSLVELGPDVEFVLLATDGLWDYIKSSEAVALVRDQLRQHGDVQVACEALGQIALDRRSQDNISIVIADLGRTNWKELPAQRPNLFLELTQAVATVGAVSLGIYISSLLALQ

SEQ ID NO: 4 - protein, 393 aa, Hordeum vulgare
EFGTRLSPRVPRLPLAAHCAGAGAGILRCCGRLAASAARCQVTASGSVAAGSSSSSELEAIRWGSAKLQGAREEMEDEVVLRPESLLHGFSFAAVLDGHAGFSTVQFLRDELFKECAAALDGGAVLNTKNLEAITDSIRRAFATVDANLSTWLQQMDKEDDSGATATALFLRNDVLVVSHIGDSCLVISRGGRPQSLTNFHRPYGNNKTSLEEVKRIRAAGGWIHDGRICGDISVSRAFGDIRFKTRKNEMLVKGVKEGRWTEKFVSRIKFNGDLIISSPDVSLVELGPDAEFVLVATDGLWDYIKSTEAVAFVRDQLRQHGDVQRACEALGEKALDRRSQDNISIVIADLGRTNWQELAVPRPNVLLELSQAIATVGAVSVGIWISWLLTLQ

SEQ ID NO: 5 - protein, 394 aa, Lycopersicon esculentum
MALSSPHLQKVLLNKICSYKITSKKSIIQKPLGGGGGGGGEGRGRCCSAIAIDAPAPFTSVSGIRWGSTMVQGPREEMEDDAVIVQSDDLDGFTYAAVFDGHAGFSSVKFLREELYKECVLALQGGLLLNRKDLNAIRKALQEAFENADRKLLNWLESSEKEDESGATATALFVGNDTLIIAHVGDSSVVLSRSGKTEILTNSHRPYGNNKVSLQEIRRINEAGGWIVNGRICNDISVSRAFGDMRFKTKKKEMLEKGVKERRWSEKFISRIQFRGDLVIASPDVLQVNLGSDAEFVLLASDGLWDYMKSDEIVDFVRNQLREHGDVQIACEALALLALDRRTQDNVSIVIADLGRTDWRNLPVKKQNVVLELGQALVTISFVSIGIWLSTMIS

SEQ ID NO: 6 - protein, 378 aa, Zea mays
MALLSPRVPRLPPSTFTSVAGARCFGPAARCQATAAGGVAAAGPPSSELEAIRWGSAKLQGARDEMEDEVLLRPGSLLDGFSFAAVLDGHAGFSAVQFLRDELYKECAAALDGGAVLSTKNLDAITASIQRAFAAVDAKLSTWLEQTDKDDSGATATVMFLRSDVLVVSHIGDSCLVISRGGRSEALTGSHRPYGNNKTSLEEVKRIRAAGGWIVDGRICGDISVSRAFGDIRFKTQKNEMLVKGVKQGRWTDKFISRIQFKDDIVISSPDVSLVELGPDVEFVLLANGLWDYIKSSEAVAFVRDQLRQHGDVQLACEALGQKALDQRSQDNISIVIADLGRTNWKALPDERPNLFLLSQAVATVGVVSIGIWVSSFL

SEQ ID NO: 7 - protein, 348 aa, Triticum aestivum
IRWGSAKLQGAREEMEDEVVLRPDSLLHGFSFAAVLDGHAGFSTVQFLRDELFKECAAALDGGAVLNTKNLEAITDSIRRAFATMDANLSTWLQQMDKEDDSGATATALFLRNDVLVVSHIGDSCLVISRGGRPQSLTNFHRPYGNNKTSLEEVKRIRAAGGWIRDGRVCGDISVSRAFGDIRFKTRKNEFVMLVKGVNEGRWTEKFVSRGRWCTVRLFLIMQPVTCRIKFKGDLIISSPDVSLVELGPDVEFVLVATDGLWDYIKSTEAVAFVRDQLCQHGDVQVCSLSLLKDRRSQDNISIVIADLGRTNWQELPVPTPNVLLELSQAVATVGAVSVGIWISSLLT

SEQ ID NO: 8 - protein, 388 aa, Sorghum bicolor
MALLSPRVPRLPPSTFTFTFAAAAGAPCFGRAARCQATAAGGVAAAGPPSSELEAVQWGSAKLQGVRDEMEDEILLRPGSLLDGFSFAAVLDGHAGFSAVQFLRDELYKECAAALDGGAVLSTKNLDAITVSIQRAFAAVDAKLSTWLEQADKDDDSGATATAMFLRNDVLVVSHIGDSCLVISRGGRPEALTSSHRPYGNNKTSLEEVKRIRAAGGWIVDGRICGDISVSRAFGDIRFKTRKNEMLVKGVKEGRWTDKFISRIKFKDDLIISSPDVSLVELGPDVEFVLLATDGLWDYIKSSEAVAFVRDQLRQHGDVQLACEALGQKALDQRSKDNISIVIADLGRTNWKALPDERPNMFLELSQAVATVGVVSIGIWFSSFLGLQ

SEQ ID NO: 9 - protein, 384 aa, Populus balsamifera subsp. trichocarpa
MALFRPQLERFLSTKLHCGHSSTKTPAKNFFFATRPKSQCSAIAIDAPSSLTDVAGIRWGSASLQGAREEMEDDIIIRSDGLEGFSFAAVFDGHAGFSSVKFLRDELYKECVAALQGGLLLNGKDFNAIRKALEEAFERVDAKLLDWYACRDGVQDESGSTATAMFIGNEKLVISHIGDSSVVLSRSGKMEVLTDAHRPYGSNKISLQEIKRIREAGGWIVNGRICGDIAVSRAFGDMRFKTKKNEMLEKGVKEGRWSEKFSSRVQFNGDLVIASPEVYQIAFGSDAEFVILASDGLWDYMNSLDAAAFVRNQLQKHGDVQLACEELARKAIDRRTQDNVSIIIADLGRTDWQNLPLQQQNVVLELGQAFATIGIVTLGIWMSS

SEQ ID NO: 10 - protein, 390 aa, Ricinus communis
MALFSPQLERFLLSKLHYGSSNFKTTSTKNLNNFVPVRPISQCSAIAIDAPSSLTDVAGVRWGSASLQGAREEMEDYIIVRSDGLDGYSFAGVFDGHGGISSVEFLRDELYKECVAALQGGLLLSGKDFNATRKALTEAFENVDKKLLNWLETIGEEDESGSTATVMFIGNNMLIVSHIGDSCLVLSRSGKAEVLTESHRPYGSNKVSLQEIKRIREAGGWISNGRICGDIAVSRAFGDIRFKTKKNEMLQKGVKEGRWSEKFISRVQFNGDLMTASPDVFQVALGSDAEFIMLASDGLWDYMNSSDAVSFVRNQLRQHGDVQLACEELAQAALDLRSQDNVSIIIADLGQTDWRNLPLQKQNILLEFGQAIATIGVVSLGIWLSSQLSL

SEQ ID NO: 11 - protein, 399 aa, Picea sitchensis
MELLSLVYYNTFMPNCSGVCVNKCTVQRRSSMECRKRGGVQVAGVAARPVQVDLSASDTSKEGSIKWGWVGIQGPRTEMEDAIVLRVDGLNNFTYAAVFDGHAGFASAKFLKDELYKECAIALQDGLLLESNDLGATREALRKAFIETDRKLISWLEEVNDGVDSGSTATVMFVGGGRLIISHIGDSSVVLSRSGKPMEITSAHRPYGNSKVSLDEIRRIKAAGGWVTNGRICGDISVSRAFGDMRFKTKMKDMLEEGIREGKWTGKFASRIKLNGDWVTAIPDIYHADLGPDIEFIIVASDGLWDCMNSSDAVKFVRSQLRQHRDVQCACEALANAALKRNTQDNVSIIIADFGRVPQEDGPEKEQNFGSEIVQATATVGIVTLGIWLSSLVGQELGR

SEQ ID NO: 12 - DNA, 1167 bp, Arabidopsis thaliana
atggcgcttctgaggccgcatcttcatcgctttcactccaacactctccgccactcagcctatccttccgccgacgctggcggtggccttgtcgtctatcccacgtatggtcgtcaccgttgctccgcgattgcgatcgacgctccgtcgtctctgactggcgtgacgccgattcgttggggttacacgagtgttcaaggattccgagatgagatggaagacgacatcgtaatccgttctgatgctgtcgatagcttctcctacgcagcggtcttcgacggccacgctgggtcgtcctctgtcaaattcctcagggaagagctgtataaagagtgtgttggtgcattgcaagctggatcgttgttgaatggaggcgatttcgcagcaatcaaggaagctttgattaaggcctttgaaagcgttgaccgcaatttgctcaaatggctagaagctaacggtgacgaagaagatgaatcaggttcaacagccactgtaatgatcatcagaaatgatgtttccttcattgctcatattggtgattcgtgtgcagtcttatctcgatctgggcaaatcgaggaattgactgattatcaccgtccatatggaagcagtagagcagctattcaggaagtgaaaagggtcaaagaagcaggtggatggattgtcaatggaaggatttgtggagacatagctgtatctcgtgcttttggtgacatccggtttaagacaaagaagaatgatatgttaaagaagggtgttgatgaaggaagatggtccgagaagtttgtttccagaatcgagtttaaaggggacatggttgtggcgactccagatatcttccaagtacctctgacatctgatgtggaattcattattttagcttctgatggattatgggactacatgaagagctcagatgtggttagttatgttagggatcagctccggaaacatgggaatgtgcagcttgcttgtgaatctcttgcgcaagtagctttggaccggcgatctcaggacaacattagcatcattattgctgatctaggaagaacagagtggaagaatctccccgcgcagcgacaaaacgtagtggttgaactggttcaggctgcaacgactatcggtttagttactgttggcatatggatgtcttcacatctatcttaa

SEQ ID NO: 13 - DNA, 1161 bp, Vitis vinifera
atggctttgctgagccagcagttgcagagattctttctcacgacactcaactgtacatccaacttgaaggccaccaagaagaacaacagtgttgccctcaaggagagggcaagatgctgctcagccatagccattgatgccccttctttttcgggtgtggcgggaatcaggtggggtttgtgccgcctccaagggcccagagaggagatggaggacgaggctgttgttcggtctgatggccttgatgggttctcctttgccgcggtttttgacggtcatgccggcttttcttctgtcaagttcctcagggatgaattgtacaaagactgtgttgcagctctgcaaggcgggttgctcctgagtgggaaaaatttcaacataattcgagaggctttggagaaggcttttgaaagtgctgatgcaaaattattgaattggcttgagacgactggggaggatgtcgagtctggttcaacagccactgtgttgttgattggagacgatatggtgttcatctcacatgttggtgactcatgtgtggttctctctcgctctggaaaagcagaagaattgaccaatcctcataggccttatggaagcaacaaaagttcgcttgaagaaatcagaagaatcagagaagcaggtggatggattgtcaatggaaggatttgcggagacattgctgtgtcccgctcttttggtgacatgaggttcaagacaaagaagaacgagatgctggagaagggattggaggaagggagatggtcccaaaaatttgtttctcgtgtacaattcactggagacttggtcgttgcatctcctgatgttttccaagtagctcttggatcagatgcagaatttttactgttagcatctgatggcttatgggattatatgaacagctcagaagcggtaacctttgttaggaatgaacttcgacaacatggagatgttcaggtagcttctgaagcacttgcacgtgcagcactggaccgacgcacacaagataatgtcagcatcatcattgcagatttagggcggacagactggcaaagtttgccactgcagcaacaaaatttatttctcgaattggtgcaagctcttggtaccattggcattgtgacgcttgggatttggatgtcatctcagtttagtttatga

SEQ ID NO: 14 - DNA, 1170 bp, Oryza sativa
atggcgctgctgagcccgcgtgtgccgcggctgccgctcgcctccgcctccgccgctggagcggggctccggtgctgcgtcagcggcggcagggccgggtcggcggcgtggtgccacgcgtcggcggccggcagcgtggcgtcgtcgtcgtcggagctggaggccatccggtgggggaccgccaagctgcagggcgcgcgcgacgagatggaggacgaggtcgtcctccgccccggctccctcctcgacggcttctccttcgccgccgtcttcgacggccacgccggattctccgccgtcgagttcctcagggacgagctgtacaaggaatgcgcggcggcgttggacggcggcgcggtgctgagcaccaagaacctcgacgccatcaccgactcgatccagcgcgccttcgccaccgtcgacgccaacctctccacctggctcgagcagatggacaaggaagatgagtccggcgccacggccacggccatgttcctcaggaacgatgtccttgttgtctcgcacattggcgactcctgcttgcaggtcgtatcacgcggtgggaggcctcaagctgtgaccaacttccaccgaccgtacgggaacaagaaagcgtctcttgaagaggtgaagcggatcagagcagcaggtggatggattgttgatggacgcatatgtggagaaatttctgtgtctcgtgcttttggagacattagatttaagacacggaagaatgagatgttggtaaaaggagttaaagaagggagatggactgaaaagtttatctcacgaataaattttaaaggagatttaatagtctcttcacctgacgtatccctggtcgaacttggaccagatgtggaatttgttttgctagcaacagatggtctttgggattacattaaaagctctgaagcagtagcattagttagggatcaactacggcaacatggggatgtccaggttgcctgtgaggcacttggtcagatagctctggatcgtcggtcgcaagacaacattagtatagtcatagctgacttggggaggacaaactggaaggagctgcctgctcaaaggccaaatttattcttggagttaactcaagcagttgcaacagttggggccgtctcattagggatttatatttcatcgctccttgcgttgcagtag

SEQ ID NO: 15 - DNA, 1182 bp, Hordeum vulgare
gaattcggcacgaggctgagcccgcgtgttccgcggctgccgctcgccgctcactgcgccggcgccggcgccggcatcctccggtgctgcggccggcttgctgcctccgctgcgcgatgccaagttacggcgtcgggaagcgtggcggcgggctcctcgtcgtcgtcggagctggaggccatccggtggggcagcgccaaactgcagggagcccgcgaggagatggaggacgaggtcgtcctccgccccgagtccctcctccacggcttctcattcgccgccgtcctcgacggccacgccggtttctccaccgtccagtttctcagggacgagctgttcaaggaatgcgcggccgcgttggacggtggcgcggtgcttaacaccaagaatctcgaggccatcacggactcgatcaggcgtgcctttgccaccgtggacgccaacctctccacctggcttcagcaaatggataaggaagacgactctggtgctactgccactgccctttttctgaggaatgatgtccttgttgtctcgcacattggtgactcttgcctggtcatatcacgtggtggaagacctcagtctctgaccaattttcatcgaccatacgggaacaacaaaacatcccttgaagaggtcaagcggattagggcagcaggtggatggattcatgatggacgcatatgtggagacatttctgtatctcgtgcttttggagacataagatttaagacacggaagaatgagatgctagtgaaaggagttaaagaaggaagatggactgaaaagttcgtttcacgaataaaatttaatggggatctaataatctcttcacctgacgtatctctggtagagcttggaccggatgcggaatttgttttggtagcaactgatggtctttgggattacattaaaagcactgaagctgtagcttttgtcagagatcaactacgtcaacatggagatgtccagcgggcttgtgaggcgcttggggagaaagctctggatcggcggtcacaagacaatattagcatagtcatagctgacttgggaaggacaaactggcaggagttggccgttccaaggccaaacgtgttgttggagctaagtcaagcaattgcgactgtaggggctgtatcagtaggaatttggatttcatggcttctcactttgcaatag

SEQ ID NO: 16 - DNA, 1388 bp, Lycopersicon esculentum
cttacactaacttcagtctggtgagaaccaagactcctccatatttcatcaatggccttatctagtccccacttgcaaaaagttcttttaaacaaaatttgcagttacaaaattacatccaaaaagagcataatacagaagcctcttggaggaggtggaggtggaggtggtgagggcagaggaagatgctgctctgcaattgcaatcgacgcgccagctccatttaccagtgtttctggcatcagatggggttcaactatggtccaaggtccgcgtgaagagatggaagatgatgctgttatcgttcaatcggatgatcttgatggcttcacttatgctgctgtttttgatggccatgctggcttctcctctgtcaagttccttagggaagagctgtataaagaatgtgttctagctttacaaggagggctgctattgaacagaaaggacctaaatgcaatcaggaaggcactacaagaagcttttgaaaatgcggataggaaactcttgaactggcttgagagtagcgagaaggaagatgaatctggtgcgacagctactgctctatttgttgggaatgatacattgatcatcgcacatgttggagattcatctgtggtgttgtcccgatctggaaaaacagagatactaaccaattcacacaggccgtatggaaacaacaaggtttctctccaagaaatcagaagaataaatgaagcaggtggatggattgtcaatggaagaatttgtaatgatatttcagtatcccgtgcttttggtgatatgagatttaagacaaagaagaaagagatgctggagaaaggggttaaagaacgcagatggtctgaaaagttcatttcacggattcagttcaggggagacttggttatagcatctccagatgttttacaggtgaatcttggatcagatgccgaatttgtactattagcatctgatggattatgggattacatgaaaagcgatgaaatagtagatttcgttaggaaccagcttcgagagcatggtgatgttcagatagcatgtgaagctcttgcactgttagccttggatcgacggacacaagataatgtcagcattgtcattgctgatttggggaggacggattggcgaaatttaccagtaaagaaacagaatgttgtgttagaactgggacaagctttagtgacaatcagttttgtgtcaattggaatatggctgtcaacaatgatttccccttagaagtttgatagtaatattagttaattgtatatagtatttttgtatgaaaaggaacataaagtttcaattatgccttgtacatttacctgtcaaggtaattaagttatgtagcattttagatagtatattctaaaaaaaaaaaaaaaaaa

SEQ ID NO: 17 - DNA, 1389 bp, Zea mays
atccctcccaactccagcgccgctaggggtcaaatcgaaaaatggcgttgctgagcccgcgcgtgccgcggctgccgccctccaccttcacctccgttgccggtgcccggtgctttggccctgccgcgaggtgccaggcgacggcggcgggcggcgtggcggccgcggggcccccgtcgtcggagctggaggccatccggtggggcagcgccaagctgcagggcgctcgcgacgagatggaggatgaggtcttgctccgcccgggctccctcctcgatggcttctccttcgccgccgtcttggatggacacgccggcttctccgccgtccagttcctcagggatgagctgtacaaggagtgcgcagctgcgttggacggcggcgcggtgctcagcactaagaatctcgacgctatcacggcctccatccagcgtgccttcgccgctgtcgacgccaagctctccacctggctcgagcaaacggacaaggacgactctggtgctacggccactgtcatgtttctcaggagcgatgtgcttgttgtctcgcacattggggactcttgcttggtgatatcacgtggtggaagatctgaagctttgactggctctcatcgaccgtatgggaacaacaaaacgtcccttgaagaggtcaagaggatcagagcagcaggtggatggattgttgatgggcgcatatgtggagacatatctgtatctcgtgcttttggggacataagatttaagacacaaaagaatgaaatgctggtgaaaggagttaaacaaggaaggtggactgataagttcatctcacgaatacaatttaaagatgatatagtaatctcgtcacctgatgtatctctggtagagcttggaccagacgtggaatttgttctgttggcaatggtctttgggactacataaaaagctctgaagctgtagcttttgtcagagatcaactccgtcagcatggtgatgtccagttggcctgcgaggcacttggtcagaaagctctggatcaacggtcccaagacaatattagcatagtcatagctgacttggggaggacaaactggaaggcgttgcccgatgaaaggccgaacttgttcttgtaactaagtcaagcagtcgcgactgtaggggttgtctcaataggaatttgggtctcgtcgttccttgggttacagtagaactagctactccgtccggatgtgtatattacctactgagtttacatctttctgctagcagtagcagctgcaaaatatatgtatattcgtatatagtgtgtgaattgatagggtaatctgtttgggtaatactgagtgaacgtgtgaagtactaaaaaaaaaaaaaaaaaaaaaaaaaaaaaacaaaaaaaaaaaaaaaa

SEQ ID NO: 18 - DNA, 3120 bp, Triticum aestivum
ggctgagcccgcgtgttccgcggctgccgctcgccgcccactccgccggcgccggcgccggatccctccggtgctgcggccgggttgctgcctccgctgcgcgatgccaagtgacggcgtcgggaagcgtggcggcgggctcctcgtcgtcgtcggagctggaggccatccggtggggcagcgccaagctgcagggagctcgcgaggagatggaggacgaggtcgtcctccgccccgactccctcctccacggcttctccttcgccgccgtcctcgacggccacgccggtttctccaccgtccagttcctgagggacgagttgttcaaggaatgcgcggccgcgttggacgggggcgcggtgcttaacaccaagaatctcgaggccatcacggactcgatcaggcgtgcctttgccaccatggacgccaacctctccacctggcttcagcaaatggataaggaagacgactctggtgccacggccacggccttgtttctgaggaacgatgtccttgttgtctcgcacattggtgactcctgcttggtcatatcgcgtggtggaagacctcaatctctgacaaattttcatcgaccatacgggaacaacaaaacatcccttgaagaggtcaagcggattagggcagcaggtggatggattcgtgatggacgcgtatgtggagacatttctgtatctcgtgcttttggagacataagatttaaaacacggaagaatgagttcgtctctcctactccctgctcttttacacacaattaatcactccatctgtcacattattcccaaataacattcctgctatttttaacatctcgtggtgttatgcaggatgctagtaaaaggagttaacgaaggaagatggactgaaaaattcgtttcacggtatagcgctgaataaagttatgttctttgttattttcttaactgcctgtgttttcatttccttgtttttgtatcaataaattaaattctcaaacaaaccaagttaggcttttctgaaacaataaggctatttgttttaggagtgtttttgctttcaattatgtgccaaaacttttttgggtggaatatgtccaaaaacacgctctttcagtagtttctactttcatgcaaagctgaaacctttgtatactggtttcatctctaaagggggggactagcagttgaatccgtatctattaagtgcacctactttcaaacgaccttacatttttttccactgttgacaaatataactgtgctcccttttcaattttttcctggtggatgaaatctaatgtttccataatttggcagatttgcttggaattttttttggcttctatcttgaacatgtaattgctcattattcaaaacctctctctaccatgcttgatagtagttgctaattagcagctgaatccgtatctattaagtgcagctactttccaacgaccttccatttttctccactgttgacaaatataactgtgcttttttttttcttttcctggtggatgaaatgtagtgtttccaaaatttggcaggtttgcttgttttttttttgacttctatacggctcattgttcacaacctctttcttgaagtttcaataaaaaaaactcttgcttgaaggtttgcacgagattttataatagctcatcgtttgtcggaccacagatgatttgcttatctgaggagagtaagctttctgttgaaacattcttttttcgatctgataaaatattttatctgcaccttgagttcttgttataactggaatgatcattttatgatcagggatttgatgaaaattttccattattatggttgaaaactatcctgaattatatattctcaatagttatctgcttatccccacacatttaatgtatatccaacttctgtacatgcttttggttgctactgcacagtatgagatgactggtataataagaaacgacatgtccaattgtgtaccttaatatcctacaacaaattacacagataacattaccatgttagatggtgaatattatagtttcaaccataactcttgccatgaaatatcatgtcgccaaagctgagcctactgtggtttacagcacttttggtagatggtgtactgtacgttaactttttttgattatgcaacctgttacgtgcagaataaaatttaaaggggatctaataatctcttcacctgacgtatctctggtggagcttggaccggatgtggaatttgttttggtagcaaccgatggtctttgggattacattaaaagcactgaagctgtagcatttgtcagagatcaactatgtcaacatggagatgtccaggtatgcagtctctctctgcttaaataaatgatactcactatatgccttttacagtacagtacatttttgtatgcttgcttttttctcatttggttgtttgtagcgggcctgtgaggcgcttggggagaaagctctggtattgttgtttgccaccttcatatcttccattcattgtttcctttccacctttatattcggcgtttattaaacctcattacaataaactatgttctgattttcctttaaaaaaattgattacaggatcggcggtcacaagacaatattagcatagtcatagctgacttggggaggacaaactggcaggagttgcccgttccaacgccaaacgtgttgttggagctaagtcaagcagttgcgactgtaggggctgtatcagtaggaatttggatttcatcgcttctcactttgcaataggcctagttaaggtcgggggtgtacattttcacatcttcggctccagctgcaaatatatatgtatatccttataggtaattagtttcctgattaaactgttcagtggtatctggactgatgaaaagaagaagaaaaacttgtgctccagctgctatctggcagcgtactaggatagggacctgtgagacgcccgtctaaactttccttttctctttcccgttttattgggggatataaaaccttatatttggcacgcgaccggcaacatttgataccatcactcctttctaaaatgaggcacttctaaattttgcggcaaaaaaaaaaaacaaaaaaaaaaaaaaaacga

SEQ ID NO: 19 - DNA, 1167 bp, Sorghum bicolor
atggcgctgctgagcccgcgcgtgccgcggctgccgccctccaccttcaccttcaccttcgccgccgcggccggtgccccgtgctttggccgtgccgcgaggtgccaggcgacggcggcgggcggcgtggcggccgcggggcccccgtcgtcggagctggaggccgtccagtggggcagcgccaagctgcagggcgttcgcgacgagatggaggatgagatcttgctccgcccgggctccctcctcgacggcttctccttcgccgccgtcttggatggacacgccggcttctccgccgtccagttcctcagggacgagctgtacaaggagtgcgcagccgcgttggacggcggcgcggtgctcagcactaagaacctcgacgctatcacggtctccatccagcgtgccttcgccgctgtcgacgccaagctctccacctggctcgagcaagcggacaaggacgacgactctggtgctacggccactgccatgtttctcaggaacgatgtccttgttgtctcgcacattggggactcttgcttggtgatatcacgcggtggaagacctgaagctttgaccagctctcatcgaccatatgggaacaacaaaacgtcccttgaagaggtcaagaggatcagagcagcaggtggatggattgttgatgggcgcatatgtggagacatatctgtatctcgtgcttttggggacataagatttaagacgcgaaagaatgaaatgctggtgaaaggagttaaagagggaaggtggactgataagttcatctcacgaataaaatttaaagatgatctaataatctcgtcacctgatgtatctctggtagagcttggaccagatgtggaatttgttctattagcaactgatggtctttgggattacataaaaagctctgaagctgtagcttttgtcagagatcaactacgtcaacatggtgatgtccagttggcctgtgaggcacttggtcagaaagctctggatcaacggtcaaaagacaatattagcatagtcatagctgacttggggaggacaaattggaaggcgttgcccgatgaaagaccaaacatgttcttggaactaagtcaagcagtcgcgactgtaggggttgtctcaataggaatttggttttcgtcgttccttggattacagtag

SEQ ID NO: 20 - DNA, 1152 bp, Populus balsamifera subsp. trichocarpa
atggcattgtttaggccacagttggagagattcctatcaaccaaactccattgtggccactccagtaccaaaacccccgctaaaaacttcttttttgctactaggcctaaaagccagtgttctgctatagcaatagatgccccatcttctttaactgatgttgctgggatccggtggggttctgcaagcttgcaaggcgcacgtgaagagatggaggatgacatcataattcgatcagatggccttgaaggattctcctttgctgctgtttttgatggccatgctggtttctcttctgtgaagttcctcagggatgagttgtacaaggagtgtgtagctgctttacaaggcgggttgctattgaatggaaaagatttcaatgctattagaaaagcattagaagaggcttttgaaagggttgatgcaaaattgctagattggtatgcctgtagagatggtgtgcaagacgaatctggttcgacagcgactgctatgttcattggaaacgaaaagctagtcatttcgcacattggtgattcatctgtggtcctttctcgttctggaaaaatggaagtgttgactgatgctcatcgaccttatggaagcaacaaaatctctcttcaagaaatcaaaagaatcagagaagcaggtggatggattgtcaatggaaggatttgtggagacatagccgtatctcgtgcttttggtgacatgcggttcaagacaaaaaagaatgagatgctggagaaaggagtcaaggaagggagatggtctgaaaaatttagttctcgagtacaattcaatggggacttggtcattgcatctccagaagtttaccaaatagcatttggatcagatgcagagtttgtaattctagcatctgatggtttatgggattacatgaacagcttagatgcagctgcttttgtcaggaaccaacttcaaaaacatggagatgttcagctagcttgcgaagaacttgcacggaaagctattgaccggcggacacaagacaatgttagcattatcattgctgatttagggcggacagactggcaaaatctgcctctccagcaacaaaatgttgtgcttgaactgggccaagcttttgctaccattggtattgtcacacttggaatatggatgtcatcc

SEQ ID NO: 21 - DNA, 1173 bp, Ricinus communis
atggcattgttcagtccccagttggagagatttctattaagcaagctccactatggcagctccaactttaaaaccactagtaccaagaacttgaacaactttgttccagtgagaccaataagtcaatgctctgccattgcaatagacgcaccatcgtctctaactgatgttgctggggtccgttgggggtctgcgagcttgcagggcgcacgtgaagagatggaagattatattattgttcggtcagatggccttgatgggtactcttttgctggggtttttgatggacatggtggtatctcttctgtcgaattcctcagggatgagctgtacaaggagtgtgttgcagctttacaaggtggcttgctattgagtggaaaagatttcaatgccacaagaaaagcattaacagaggcttttgaaaatgtggataaaaagttgttaaattggcttgaaacaattggtgaggaagatgaatcaggttcaacagccaccgttatgttcattggaaacaatatgcttatcgtttcgcatattggtgactcatgcctggttctgtctcgttctggaaaagctgaagttttgactgaatcccatcgaccttatgggagcaacaaggtttctcttcaagaaatcaaaagaatcagggaagcaggtggctggatcagcaatggaagaatctgtggagatatagctgtatcacgtgctttcggagatataaggttcaagacaaagaaaaacgagatgctgcagaaaggagttaaggaagggagatggtctgaaaaatttatttctagagtgcaattcaatggggacttgatgactgcatctccagatgtatttcaagtagctctgggttcagatgctgaatttataatgttagcatctgacggtttatgggattacatgaacagctcagatgcagtttcttttgtcaggaaccaacttcggcaacatggagatgttcagctagcttgcgaagaacttgcacaagcagctttggatctacgttcacaagacaatgttagcatcattattgctgatttagggcaaacggattggcgaaatttgccactacagaaacaaaatatattgctcgagttcggccaagctattgccaccatcggtgttgtgtcacttggaatttggttgtcatctcagctttctttataa

SEQ ID NO: 22 - DNA, 1200 bp, Picea sitchensis
atggaattattaagcctcgtttattataatactttcatgccaaattgttcaggagtttgtgtgaacaaatgcacggttcagaggagaagttccatggaatgcagaaaacgaggcggtgtacaggtggctggagtagcagcgagacccgtacaggtggatctttcagcgtctgacacgtcaaaagaaggaagcattaaatggggatgggttggaattcaaggccccagaacagagatggaagatgccatcgttttacgagtggacgggcttaataatttcacttacgcagcagtctttgacggccatgctggattcgcttctgctaaatttttgaaggatgaattgtacaaagaatgtgcaatagctttacaagatggcttacttttggagtccaacgacttgggtgctacaagggaggcattaaggaaagcttttatagaaactgataggaagctgatatcctggctcgaagaagtaaatgatggggttgactcgggctcaacagcaactgttatgtttgtaggaggtggcagattgataatttcacatattggtgattcttctgtggtactttcccgttcaggaaagccaatggagattacgagtgctcaccgcccatatggaaatagcaaagtttcccttgatgaaatacggcgtataaaagcagcaggtggttgggttacaaatggtcgaatttgtggagacatatctgtttcacgtgcatttggggatatgcgattcaagacaaagatgaaagatatgttggaggaaggtattcgagaagggaaatggacagggaaatttgcatctagaatcaagcttaatggtgactgggttactgccatacctgacatttatcatgcagatttgggccctgatatcgagtttattatagttgcctctgatggcctttgggattgcatgaacagttcggatgcagtaaagtttgttaggagtcaacttcgacaacatcgtgatgtgcagtgtgcttgtgaagcacttgccaatgctgctctgaagagaaatacacaggataatgttagtattatcatcgcagactttgggagagttccccaggaggatggaccagagaaagagcaaaattttggaagtgaaattgtgcaagctactgcaacagttggtattgtaaccttaggaatttggctatcatctctagttggccaagagcttggcagataa

SEQ ID NO: 23 - protein, 387 aa, Cucumis sativus
MALLSPRLHRFRLTKLHHVSASKPTKATKLTLRKNSRCCSAIAIDAPSSLTDAAGIRWGSTTFQGLREEMEDDAVVRSDGFNDFLFAAVFDGHGGYSSVKFLREELYKDCVAALQGGQLLNGGDFEVIKAALEKAFDDTDKRLLLLLEAAGEEDESGATATVAFIRNDVLFISHLGDSCVVLSRSGGAQVLTSSHRPYGNNSTSLQEIRRIREAGGWIVNGRICGDISVSRAFGDIRFKTKKSEMLQKGVEEGRWSEKFISRVQFNGDLVTASPEIFQVTLGSNAEFVLMASDGLWDYMNSSDAVMFVRNELRQHGDVQLACEALAQAALDKGSQDNVSIIMADLGRTDWQNLPLQQDNVIFELAQALATMGLVSLGIWWASSSFSL

SEQ ID NO: 24 - protein, 301 aa, Brassica rapa
SFAAVFDGHAGSSPVKFLREELYKGCVEALQVGSLLKGGDFGAIKGALVKAFESVDQNLLKWLETKGEEDESGSTATVMLIRNDVSYFAHIGDSCAVLSRSGQIEELTDSHRPYGSSKAAVQEMKRIKDAGGWIVNGRICGDIAVSRAFGDIRFETKKHEMLKKGVDEGRWSEKFVSRIEFKGDMVVATPDIYQVPLTSDVEFIILASDGLWDYMKSKDVVSFVREQLREHGNVQLACEALAQVALDRRSEDNISIIIADLGRTEWKKLPVQRQNMVVELVQAAATVGIVTVGIWMTSLLS

SEQ ID NO: 25 - protein, 220 aa, Brassica napus
RCSAIAIDAPSSLAGVTPIRWGYTSVQGFREEMEDDIVIRSEALDSFSFAAVFDGHAGSSSVKFLREELYKGCVEALQVGSLLKGGDFGAIKGALVEAFESVDQSLLKWLETKGEEDESGSTATVMLIRNDVSYFAHIGDSCAVLSRSGQIEELTDSHRPYGSSKAAVQEMKRIKDAGGWIVNGRICGDIAVSRAFGDIRFKTKKHEMLKKGVDEGRWSE

SEQ ID NO: 26 - protein, 288 aa, Nicotiana tabacum
PLRPGEELYKECITALQGGLLLEKRDLNAIQKALQEAFENADRKLLNWLESSGKEDESGSTATVLFVGNDTLIIAHVGDSSVVLSRSGKMEVLTNAHRPYGSNKVSLQEIRRINEAGGWIVNGRICGDISVSRAFGDMRFKTKKNEMLEKGIEEGRWSEKFVSRIQFKGDLVTASPDVLQVALGSDTEFVLLASDGLWDYMKSAEVVDFVRNQLREHGDVQVACEALARTAMDRRTQDNVSIVIADLGRTDWRNLPVQKQNIVFELGQALITISFVSLGIWLTTVISP

SEQ ID NO: 27 - protein, 234 aa, Raphanus sativus
NTLHQSAPHTKPISDAGGGGLVVYPTYGRRRRHHRCSAIAIDAPSSLAGVTPIRWGYTSVQGFRDEMEDDIVIRSEAIDSFSFAAVFDGHAGSSSVKFLREELYKECVGALQVGSLLSGGDFVAVKEALVKAFESVDQSLLKWLETKGEEDESGSTATVMLIRNDVSYFAHIGDSCAVLSRSGQIEELTDSHRPYGSTKAAVQEMKRIKDAGGWIVNGKICGDIAVSRAFGDIW

SEQ ID NO: 28 - protein, 251 aa, Citrus sinensis
FENVDMKLLNRLEMNAEEDESGATATVMFIGRDILFISHVGDCCVVLSRTGKADVLTNPHRPYGSGQVSLQEIRRIREAGGWISNGRICGDIAVSRAFGDIRFKTKKNEMLKKGVNEGRWSEKFVSRVQLNKDLVIASPDIFQVSLGSDAEFVLLASDGLWDYMNSSDAVKFVRNQLQQHGDVQLACEALAQAALDQHSQDNISIVIADLGQTDWRNLPLQQQNVVCELAQAFATIGIVSLGIWMSSLLSV

SEQ ID NO: 29 - protein, 264 aa, Cichorium intybus
GESFQECVKALQGGILLSKKDTTGIEKALQEAFENADTKLLNWLEMNGEDDESGATATTMFLGNNMMFISHVGDSCAVLSQSGKAQELTDSHRPYGRSKVSLQEIKRIREAGGWISDGRICGDISVSRAFGDMRFKTKKKEMLEKGVAEGRWSEKFVSRVRFVGDLVIATPDIYQVALGPEAEFVLLATDGLWDYIKSSDAVNFVRNQLREHGDVQAASEALAQMALDQYSQDNVTIVIADLGRTDWQNLPIQQQNFVYELIQA

SEQ ID NO: 30 - protein, 197 aa, Raphanus raphanistrum
TKGEEDESGSTATVMLIRNDVSYFAHIGDSCAVLSRSGQIEELTDSHRPYGSTKAAVQEMKRIKDAGGWIVNGRICGDIAVSRAFGDIRFKTKKHEMLKKGVDEGRWSEKFVSRIEFKGDMVVATPDIYQVPLTSDVEFIILASDGLWDYMKSKDVVSFVREQLREHGNVQFACEALAQVALDRRSEDNISIIIAGL

SEQ ID NO: 31 - protein, 253 aa, Solanum tuberosum
EAFENADRKLLNRLESSEKEDESGATATAVFVGNDTLIIAHVGDSSVVLSRSGKAEILTNSHRPYGNNKVSLQEIRRINEAGGWIVNGRICNDISVSRAFGDMRFKTKKKEMLEKGVKERRWSEKFISRIQFRGDLVIASPDVLQVNLGSDTEFVLLASDGLWDYMKSDEIVDFVRNQLREHGDVQIACEALARLALDRRTQDNVSIVIADLGRTDWQNLPVKKQNVVLELGQALVTISFVSIGIWLSTMIFP

SEQ ID NO: 32 - protein, 257 aa, Aquilegia sp.
NALEEAFMNADSKLINWLEETGKQTESGSTATVMFANNEMLIISHVGDSSVVLSCSGKAEILTNPHRPYGNNKVSLQEIRRIREAGGWIVNGRICGDISVSRAFGDMRFKTKKNEMLAQGVEEGRFSAKFVSRVQFKDDLVIASPDIYQVALASDAEFILLASDGLWDYVNSLDAVSFVRNQLRQHGNVQRACEALAQTALDRRSEDNISIIIADLGRTNWESLPPQQQNFVWEVSQAFGTLALVSLGIWASSFLSL

SEQ ID NO: 33 - protein, 289 aa, Citrus clementina
  misc_feature (206)..(206): Xaa can be any naturally occurring amino acid
LNQQLQRCLLTRLNYNFKPTVKNNFVNTTTARAKSQCYCSAIAIDAPSSLSGVAGIRWGSVSLQGLREEMEDGAVIQSDGLDGFSFAAVFDGHGGVSTVKFLRDELYKECVAALQGGLLLSGKDFDAIKKALQEAFENVDMKLLNRLEMNAEEDESGATATVMFIGRDILFISHVGDCCVVLSRTGKADVLTNPHRPYGSGQVSLXEIRRIREAGGWISNGRICGDIAVSRAFGDIRFKTKKNEMLKKGVNERRWSEKFVSRVQLNRDLVNGSPKIFKYLLGQMQNLYW

SEQ ID NO: 34 - protein, 290 aa, Phaseolus vulgaris
PHMQRFLLCKLHTRSTSKTTHKNTFTTTTRGRCCSAIAIDAPSSLTEVPGVRWGSIALQGLREEMEDDIIVRPDGLQGFSFAAVFDGHGGFSSVEFLRDELYKECVEALQGGLLLVQKDFKAAKGALQEAFLKADTRLLKRLEMNGEEDESGATATAVFIGGDELLISHIGDSSAVLCRSGKAEVLTSPHRPYGSNKTSLNEIRRIREAGGWISNGRICGDIAVSRAFGDMRFKTKKNEMLQKGVQEGKWSAKFISRVQLNNDLVVAYPDIYQVALGSDAEFIVLATDGL

SEQ ID NO: 35 - protein, 230 aa, Vigna unguiculata
GATATAIFIGDDELLISHIGDSSAVLCRSGKAEVLTSPHRPYGSNKTSLDEIRRIREAGGWISNGRICGDIAVSRAFGDVRFKTKKNEMLQKGVQEGKWSAKFISRVQLNNDLVVAYPDIYQVALGSDAEFVVLATDGLWDYMSSSEAVSFVRDQLRKHGNIQLACESLAEAALDRRTQDNVSIIIADLGRTDWQNVPLERQNTIVELVQALATIGIVSFGIWFSSQLSL

SEQ ID NO: 36 - protein, 237 aa, Triphysaria pusilla
QLVREDESGSTATTIFLCDDVLVVSHVGDSCVVLSRDGKVEVLTDSHRPYGNNKASLQEIRRIREAGGWIVNGRICGDISVSRAFGDIRFKTKKNEMVAKGVEEGRWPAKFASRLKLKGDLVTACPDVFQVSIGSDAEFIVLASDGLWDYINSSDTVKFIRNHLRQYGDVQMASEALAQMALNQNSQDNISIIIADLGRTDWKKLPVQQQNIAVELVQALNTLSLVSLGIWISSLLS

SEQ ID NO: 37 - protein, 268 aa, Carthamus tinctorius
GDDAVIVASSSSNNDLDGFHFAAVFDGYAGFSSVDFLREELYKECVKALQGGLLLSKKVTEIEKALQEAFMNADAKLLNWLEMSGKDDESGATATTMFVGNDMLFISHVGDSCAVLSESGKPEVLTNSHRPYGRHKVSLQEIKRVREAGGWIVDGRICGDISISRAFGDIRFKTKKTEMLEKGVEEGRWTQKFISRIRFSGDLVIATPGIYHVTTGSDAEFVLLATDGLWDYINSSDAVNFVRNQLREHGDVQAASEALAQMALDQYS

SEQ ID NO: 38 - protein, 259 aa, Citrus reticulata
  misc_feature (229)..(229): Xaa can be any naturally occurring amino acid
HASGYCSAIAIDAPSSFSGVAGIRWGSVSLQGLREEMEDGAVIQSDGLDGFSFAAVFDGHGGVSTVKFLRDELYKECVAALQGGLLLSGKDFDAIKKGLQEAFENADMKLLNWLEMNAEEDESGATATVVFIGRDILFISHIGDCCVVLSRTGKADVLTNPHRPYGSSQVSLQEIRRIREAGGWISNGRICGDIAVSRAFGDFRFKNKKNEMLKKGVNEGRWSEKICSXSALYKDLVIASPDIFQVSLGVRCRICTVYL

SEQ ID NO: 39 - protein, 319 aa, Lactuca sativa
  misc_feature (272)..(272): Xaa can be any naturally occurring amino acid
GFSATHLLSSMALWSPRLQRFLLNNNVSKIDLKKTNSCNSNNHIIMRKWSREGSPRCCSAIAIDAPSSLSDGLGSGIRWGSAKLQGAREEMEDDAVIVGSSPHNELDGFFFAAVFDGHAGFSSVKFLREELYKECVKALQGGLLLSKKDTIGIEKALQEAFQNADAKLLNWLEMNGEDDESGATATTMFLGNNMLFISHVGDSCAVLSQSGKAQELTNSHRPYGRNKVSLQEIKRIREAGGWISDGRICGDISVSRAFGDMRFKTKKKEMLXKGVAESRWSEKFVSRMRFFGDLVIATPDIYQVTLGPEAEFCMVATDG

SEQ ID NO: 40 - protein, 230 aa, Gossypium hirsutum
  misc_feature (220)..(220): Xaa can be any naturally occurring amino acid
DDESGSTATVMLIGNEVLFISHVGDSCVVLSCAGKVQVLTDSHRPYGSNKASLQEIRRIREAGGWISNGRICGDIAVSRAFGDTRFKTKKNEMLKKGVEEKRWSEKFISRVVFNDDLVIASPDTFKVALGSDAEFVLLASDGLWDYINSSDAVAFVRNQLREHGDVQVACDVLAQAALDKGSEDNVSIIIADFGHTEWQKLPVEQQNFLFEFGQAIATVXVVSLGIWLSS

SEQ ID NO: 41 - protein, 235 aa, Helianthus annuus
FSSVNFLREELYKECVKALQGGQLLDNKDFIGIKNALQEAFHNADAKLLNWLEMTSEEDGESGATATAIFIGNDMLYISHVGDSCAVLSQSGNAEELTNSHRPYGRNKVSLQEIKRIREAGGWISDGRICGDISVSRAFGDIRFKTKKNEMLKKGVEEGRWSEKFASRMKFFEDLVIATPDIYQVALGPDAEFVLLATDGLWDYIKSSDAVSFIRNQLREHGDVQVASEALAQMA

SEQ ID NO: 42 - protein, 252 aa, Glycine max
KDELYKECVEALQGGLLLVEKDFKAIKRALQEAFLKADARLLKRLEMNGEEDESGATSTAVFIGDDELLISHIGDSSAVLCRSGKAEVLTSPHRPIGSSKTSLHEIRRVREAGGWINNGRICGDIAVSRAFGDVRFKTKKNEMLQKGVQEGRWSAKFISRVQLNNDLVVAYPDIYQVTLGSDAEFVVLASDGLWDYMSSSEAVSLVRDQLRKHGNIQQACEALAEAALDRRTQDNVSIIIADFGRTDWQNAP

SEQ ID NO: 43 - protein, 252 aa, Avena barbata
LDGGAVLNTKNLEAITESIRRAFATVDANLSTWLEKMDKEDESGATATALFLRKDVLVVSHIGDSCLQVVSRGGRPQSLTNFHRPYGHNKTSLEEVKRIRAAGGWILDGRICGDISVSRAFGDIRFKTRKNEMLVKGVKEGRWTEKFVSRIKFKGDLIISSPDVSLVELGPDVEFVLVATDGLWDYIKSTEAVAFVRDQLRQHGDVQRACEALGEKALDQRSQDNISIVIADLGKDKLERSCPYQGQTCSWS

SEQ ID NO: 44 - protein, 258 aa, Panicum virgatum
LDGGAVLSTKNLEAITASIQRAFAAVDAKLSTWLEQMDKDDDSGATATAMFLRNDVLVVSHIGDSCLMISRGGRPEALTSSHRPYGNNKTSLEEVKRIRAAGGWIVDGRICGDISVSRAFGDIRFKTRKDEMLVKGVKEGRWTEKFISRIKFKEDIIISSPDVSLVELGPDVEFLILATDGLWDYMKSSEAVAFVRDQLRQHGDVQLACEALGQKALDERSQDNISIVIADLGRTNWKALPVERPNLFLGTKSSSCDL

SEQ ID NO: 45 - protein, 213 aa, Artemisia annua
HIGDSSAVLSLSGKAEELTDSHRPYGRSKASLQEIKRIREAGGWISDGRICGDISVSRAFGDTRFKTKKNEMLEKGLAEGRWSEKFVSRIKFVGDLVIATPDIYHVTLGPDAEFVLLATDGLWDYIKSSEAVNFVRNQLREHGDVQVASEALAQMALDQYSQDNVTIVIADLGRTDWRNLPIQQQNFVFELIQAFATIGIVTIGIWMSSNASF

SEQ ID NO: 46 - protein, 234 aa, Allium cepa
SEMEDDVVMRLDGLDGFAFAAVFDGHAGFSSVEFLRDELYKECVAALQGGSLLTSKNFNAIKDGIAKAFRTVDIKLSSWLEQVGKDADSGATATAMFLGKDVLIISHVGDSSVVISRNGKAEILTGPHRPYGNNKVSLEEVKRIRAAGGWIIDGRICGDISVSRAFGDIRFKAKKNEMLRKGVEEGRWTEKFVSRIRFKGDLVTSSPDVFQLELGSDVEFVLLASDGLWDYIRS

SEQ ID NO: 47 - protein, 201 aa, Arachis hypogaea
VLSRAGKAEMLTSPHRPYGRNKASLQEIKRIREAGGWIVNGRICGDIAVSRAFGDLRYKTKKNEMLQKGVQEGRWSEKFISRVRLNNDLVVAYPDIYQVDLGSDAEFIVLASDGLWDYMSSSDAVSFVREQLQKHGNIQVACEALAQAAMDRRTQDNVSIIIADLGKTDWENVAVQQQNMVLELVQALATIGIVSIGIWFS

SEQ ID NO: 48 - DNA, 1164 bp, Cucumis sativus
atggctttgctgagtcctcgattgcatagattccggctaactaagcttcaccatgtctccgcctccaaacccaccaaagcaactaagctcactctcagaaaaaattccagatgctgctctgccatagctattgatgctccatcttctttaacagatgctgcgggaataagatggggttccactacttttcaaggcttgcgggaggaaatggaagacgatgctgttgtgcggtctgatggtttcaatgacttcttgtttgctgcagtgttcgatggccatggtggctattcctctgtcaagttcctaagggaggagctgtacaaagactgcgtggcagccttgcagggcggccaactattgaatggaggggattttgaagtgataaaagcggctttagagaaggcttttgatgatactgataagagactactcctattgcttgaggcagctggggaggaagatgaatctggtgccactgcaaccgttgcattcattcgaaatgatgtgctgttcatttcacatcttggtgactcttgtgttgtgttatctcgttctggaggagcacaggtgttgactagttctcatcggccttatggaaataacagcacatcactgcaagagattagaagaattagagaagcaggtggatggattgttaatgggagaatctgtggggacatctctgtatcacgtgcatttggtgacatacgattcaagacaaagaaaagcgagatgctacagaaaggagttgaagaaggaagatggtcagaaaaatttatttctcgtgtacagttcaatggagatttggttactgcatcaccagaaattttccaagtcacactcggatcaaatgccgagtttgtactgatggcatcggatggcctatgggattacatgaatagctctgatgcggttatgtttgttcggaatgaacttagacagcatggcgacgttcagctagcttgtgaagccctcgcccaagcagccttggacaaagggtcacaagacaatgtcagcattatcatggctgatttagggcggacagattggcaaaatttgccccttcagcaagacaatgtcatatttgaactggctcaagctttggctacgatgggactcgtttctttaggtatttggtgggcatcctctagtttctctctttaa

SEQ ID NO: 49 - DNA, 923 bp, Brassica rapa
tctccttcgccgcggttttcgacggccacgctggatcctcccctgttaagttcctcagggaggagctgtacaagggatgtgttgaggcgttgcaagttggatctttgttgaaaggtggtgactttggtgcgatcaagggagctttggtcaaggcctttgagagcgtggatcagaatctgctcaaatggctagaaacaaaaggtgaagaagatgagtctggttcaacggctactgtgatgctcattagaaatgatgtttcctactttgcacatattggggattcttgcgctgtcttatctcgatctggacaaatcgaggagttgactgattctcatcgtccatatggaagcagtaaggcagctgttcaggaaatgaagaggatcaaagatgcaggtggatggattgtcaatggaaggatttgtggagacatagctgtatctcgtgcttttggtgacatccggttcgagacaaagaagcatgagatgttaaagaagggtgtcgatgaaggaagatggtccgagaagtttgtctcgagaatcgagtttaaaggggacatggttgtcgcaactccggatatctaccaagtgccccttacatcagatgtggagtttattattttagcttctgatggactgtgggactacatgaagagcaaagatgtggttagcttcgtaagggaacagctccgagaacatggaaatgtccagcttgcttgtgaagctcttgcgcaagttgctttggaccggagatcagaggacaacatcagcatcatcatagctgatctaggaagaacagagtggaagaagctcccggtgcagcggcaaaacatggtggttgaactggttcaggcggcagccactgtgggaatagtcactgttggcatatggatgacttctcttctctcttaaagtaaatggaacatc

SEQ ID NO: 50 - DNA, 807 bp, Brassica napus
cgttgctccgcgattgcgatcgacgctccgtcgtctctcgcgggcgtgacgcctatccggtgggggtacacgagcgttcaagggtttagggaggagatggaggacgacatcgtcatccgctcggaagctctcgacagcttctccttcgccgcggttttcgacggccacgctggatcctcctctgttaagttcctcagggaggagctgtacaagggatgcgttgaggcgttgcaagttggatctttgttgaaaggtggtgactttggtgcgatcaagggagctttggtcgaggcttttgagagcgtggatcagagtttgctcaaatggctagaaacaaaaggtgaagaagatgagtctggttcaacggctactgtgatgctcattagaaatgatgtttcctactttgcacatattggtgattcttgcgctgtcttatctcgatctggacaaatagaggagttgactgattctcatcgtccatatggaagcagtaaggcagctgttcaggaaatgaaaaggatcaaagatgcaggtggatggattgtcaatggaaggatttgcggagacatagctgtatctcgtgctttcggtgacatccgattcaagacaaagaagcatgagatgttgaagaagggtgtcgatgaaggaagatggtccgagaagtttgtatccagaatcgagtttaaaggggacatggttgtcgcaactccggatatctaccaagtacctctcacatcggacgtggagttcattattttagcttctgatggattatgggactacatgaagagcaaagacgtggttagt

SEQ ID NO: 51 - DNA, 904 bp, Nicotiana tabacum
ggccattacggccgggggaggagctgtataaagaatgtattacagcattacaaggcgggctgctattggaaaaaagggacctaaacgcaatccagaaggcactacaagaagcttttgaaaatgcggatagaaaactcttgaactggcttgagagtagcgggaaagaagatgaatctggttcaacagctactgttctatttgttgggaatgacacattgatcattgcacatgttggagattcatctgtggtgttgtcacgatctggaaaaatggaggtattaacaaatgcacacaggccctatggaagcaacaaggtttctctccaagaaatcagaagaatcaatgaagcaggtggatggattgtcaatggaagaatttgtggagatatttcagtatcccgtgcttttggcgatatgagatttaagacaaagaagaatgagatgctggagaaagggattgaagaaggcagatggagtgaaaagtttgtttcgcggattcagttcaagggagacttggttacagcatctccagatgttttacaggtggctcttggatcagatactgaatttgtactattagcatctgatggattatgggactacatgaaaagtgctgaagtagtagattttgttaggaaccagcttcgagagcatggagatgttcaggtagcttgtgaagctcttgcacggacggccatggaccgacggacacaagataatgttagcattgtcatagctgatttggggagaacggactggcgaaatttacctgtgcagaaacaaaatattgtgtttgaactaggacaagctttaatcacaatcagttttgtgtcacttgggatatggctgactaccgtgatttccccttagaaatttgatttttgttagttgtatatatttatata

SEQ ID NO: 52 - DNA, 706 bp, Raphanus sativus
ggaacactctccaccaatcagcccctcatactaaaccaatctccgacgccggcggcggtggacttgtcgtctatcccacttacggtcgtcgtcgtcgtcaccaccgttgctccgcgattgcgatcgacgctccgtcgtctctcgcgggcgtaacgcctatccggtgggggtacacgagcgttcaagggttcagggatgagatggaggacgacatcgtcatccgttcggaggctatagatagcttctccttcgccgcggtcttcgacggacacgctggatcctcctctgttaaattcctcagggaagagctgtacaaggagtgtgttggagcattacaagttggatccttgttgagtggaggcgatttcgtggcggtcaaggaagctttggtgaaggcctttgagagcgtggatcagagtttgctcaaatggctagaaacaaagggtgaagaagatgaatcgggttcaacggctactgtgatgctaatcagaaatgatgtttcctactttgcacatattggtgattcttgtgcagtcttatctcgatctggacaaatagaggaattgactgattctcatcgtccatatggaagtactaaggcagctgttcaggaaatgaaaagaatcaaagatgctggtggatggatcgtcaatggaaagatttgtggagacatagctgtatcccgtgctttcggtgacatctggtt

SEQ ID NO: 53 - DNA, 914 bp, Citrus sinensis
tttttttgaataaaatgagaaccagtttattgttatgtggagattcttcttagttaactaattgactattatggtgctatactaagaatgattacatgatgtagaagaaagcgttcaattctgtgtgattatatacatttccagagacatttcatctcatactgaaagcagagatgacatccatattccaagtgacacaatgccaatcgtagcaaatgcctgggccaattcgcacacaacattttgttgctgaagtggcaaatttctccagtcagtctgccctaaatcggcaatcacaatgctgatattatcttgggaatgttggtccaaagcagcttgtgccagtgcttcacaagctagctgtacatctccatgttgttggagctgattcctgacgaatttaactgcatctgagctattcatgtaatcccatagtccatcagatgctaacagtacaaattctgcatctgacccaagagatacttgaaaaatatctggtgatgcaataaccaggtccttgttaagttgcactctagaaacaaatttttctgaccatcttccttcattaacacccttcttcagcatctcatttttctttgtcttgaaccggatgtcaccaaaagcacgagatacagcaatgtcaccacatatcctaccattggaaatccatccacctgcttctctgattcttctaatttcctgaagtgagacctggccgcttccataaggccgatgaggattagtcaatacatctgcttttccagtacgagatagaaccacacaacagtcaccaacatgtgagatgaacaaaatatcccttccaatgaacataacagtagcggttgcaccggattcgtcctcctccgcattcatttcaagcctattcaacaatttcatatcaacattttcaaaag

SEQ ID NO: 54 - DNA, 795 bp, Cichorium intybus
cggggagagctttcaggagtgtgtgaaggctctacaaggggggatattgttaagtaaaaaggacaccactggaatcgaaaaggctttacaagaggctttcgagaacgccgatacgaaacttctaaattggcttgagatgaacggagaggacgatgaatctggtgcaacagccactaccatgttcctaggaaacaatatgatgttcatctctcatgttggtgattcatgtgcggttctttcacagtctggaaaagcacaagaattgaccgattctcacaggccctatggaagaagcaaggtttcccttcaagaaatcaagcggatcagagaagcaggtggatggatttcggatggaagaatttgtggagatatttctgtatcccgtgcttttggtgacatgcggtttaaaacaaagaagaaagagatgctggaaaagggagttgcagaaggcagatggtctgaaaaatttgtatcccgggtacggtttgttggagacttggttattgctactcctgatatttatcaagttgcactggggcctgaagcagaatttgtattgttagcaactgatggcttatgggattacataaaaagctcggatgcagtcaactttgtcagaaaccaacttcgggaacatggagatgtgcaggctgcatctgaagctctagctcaaatggccttggaccaatattctcaagataatgtaaccattgtcatcgctgatttggggcggacagattggcaaaatttacctatacagcaacaaaactttgtgtatgaactgatccaagcttt

SEQ ID NO: 55 - DNA, 594 bp, Raphanus raphanistrum
ggacaaagggtgaagaagatgaatcgggttcaacggctactgttatgcttatcagaaatgatgtttcctactttgcacatattggtgattcttgtgcagttttatctcgatctggacaaatcgaggaattgactgattctcatcgtccatatggaagtactaaggcagctgttcaggaaatgaaaagaatcaaagatgctggtggatggattgtcaatggaaggatttgtggagacatagctgtatctcgtgctttcggtgacatccggttcaagacaaagaagcatgagatgttgaagaagggtgtcgatgaaggaagatggtccgagaagtttgtgtcaagaatcgagtttaaaggggacatggttgtcgcaacaccggatatataccaagtacctcttacatcggatgtggagttcattattttagcttctgatggattgtgggactacatgaagagcaaagatgtcgttagtttcgtaagggaacagctccgagaacatggaaatgtccagtttgcttgtgaagctcttgcgcaagttgctttggaccggagatcagaggataacatcagcatcatcatcgctggtctag

SEQ ID NO: 56 - DNA, 927 bp, Solanum tuberosum
caacagttaaatttggaattttccataattttagaatatactatctaaaatgctacataacataattaccttgacaagcaaatgtacaaggtataattgaagctttatgttccttttcatacaaaaatactatatacaattaactaatattactatcaaacttctaagggaaaatcattgttgatagccatattccaattgacacaaaactgattgtcactaaagcttgtcccagttctaacacaacattctgtttctttactggtaaattttgccaatccgtcctccccaaatcagctatgacaatgctgacattatcttgtgtccgtcgatccaaggctaaccgtgcaagagcttcacaagctatctgaacatcaccatgctctcgaagctggttcctaacgaaatctactatttcatcacttttcatgtaatcccataatccatcagatgctaatagtacaaattcggtatctgatccaagattcacctgtaaaacatctggagatgctataaccaagtctcccctgaactgaatccgagaaatgaacttttcagaccatctgcgttctttaacccctttctccagcatctctttcttcttcgtcttaaatctcatatcaccaaaagcacgggatactgaaatatcattacaaattcttccattgacaatccatccacctgcttcatttattcttctgatttcttggagagaaaccttgttgtttccatagggcctgtgtgaattggttagtatctctgcttttccagatcgggacaacaccacagatgaatctccaacatgcgcgataatcagtgtatcattcccaacaaatacagcagtagctgtcgcaccagattcatcttccttctcgctactctcaagccggttcaagagtttcctatccgcattttcaaaagcttctt

SEQ ID NO: 57 - DNA, 867 bp, Aquilegia sp.
tgacagtaatatgatgtaaggcacaagtatttacaagttatacacgatgtcttgtttatatgtatttacacatctgagagacattttccactttaaagagaaagaaaagatgatgcccaaattcctagtgagactagagctaaagttccaaacgcttggcttacttcccatacaaaattttgttgctggggcggtaaactctcccagtttgtcctccctaaatcagctatgatgatgctaatattatcttctgagcgccgatccagagctgtttgtgccagcgcttcacaagcacgctgaacattgccatgttgtcgaagttgattcctaacaaatgataccgcatccaaactgtttacataatcccataagccatcagatgctaataatataaattctgcatctgatgcaagagcaacttgataaatgtctggagaagctataacaaggtcgtctttgaactgcactcgagaaacaaattttgcagaaaatcttccttcctcaactccttgcgccaacatttcgttcttctttgtcttgaatcgcatgtcaccaaaagcacgagatacagagatatccccacaaatccttccattgacaatccatccacctgcttctctgatcctcctgatttcttgaagagagaccttgttgttcccatacggtctatgcgggttggtcaatatttctgcttttccagaacaggacaaaaccacacttgagtcaccaacatgtgaaatgattagcatctcattattagcaaacataactgtagctgtggaacctgattcagtttgcttcccagtctcttcaagccaattgattaacttcgaatcagcattcataaaagcctcctctaatgcattt

SEQ ID NO: 58 - DNA, 893 bp, Citrus clementina
  misc_feature (620)..(620): n is a, c, g, or t
tgctgaaccaacagttacagaggtgtctcttaacaaggctcaactataacttcaagcccaccgtcaaaaacaactttgtcaatacaacaacagcaagagctaaaagccagtgctactgctcggcgattgcaatagacgcgccgtcttcgttgagtggtgttgccggaatccgatgggggtctgtaagtttacagggcctacgtgaggaaatggaggatggtgctgttattcaatcggatggccttgatgggttctcttttgcagctgtttttgatggccatggtggagtctccactgtcaagtttcttagggatgagctatacaaagaatgtgttgcagctttacaaggaggattgctgttgagtggaaaagattttgatgccattaagaaggcattacaggaggcttttgaaaatgttgatatgaaattgttgaataggcttgaaatgaatgcggaggaggacgaatccggtgcaaccgctactgttatgttcattggaagggatattttgttcatctcacatgttggtgactgttgtgtggttctatctcgtactggaaaagcagatgtattgactaatcctcatcggccttatggaagcggccaggtctcacttcangaaattagaagaatcagagaagcaggtggatggatttccaatggtaggatatgtggtgacattgctgtatctcgtgcttttggtgacatccggttcaagacaaagaaaaatgagatgctgaagaagggtgttaatgaaagaagatggtcagaaaaatttgtttctagagtgcaacttaacagggacctggttaatggctcaccgaaaattttcaagtatctcttgggtcaaatgcaaaatttgtactggtagcctctgatggaatatgggaat

SEQ ID NO: 59 - DNA, 874 bp, Phaseolus vulgaris
ctccacacatgcaaagattcctcttatgcaagctccatactcgctccacctctaagaccacacacaaaaacaccttcaccaccaccaccaggggccggtgctgctctgccatagccatcgatgctccatcgtctttgactgaggtgcccggtgtgagatgggggtccatagccttgcagggtcttcgtgaagagatggaggacgacatcattgttcgacccgatggccttcagggcttctctttcgcggctgtcttcgatggccatggagggttttcttctgtcgagttcctcagggatgagctgtacaaggagtgtgttgaagctttacaaggtggattactattagtgcagaaagatttcaaagccgctaaaggagcattgcaggaggcatttctcaaggcagacacgaggttactaaaacggcttgaaatgaatggagaggaggatgaatcaggtgctacagcaactgccgttttcatcggaggtgatgagcttctaatttcacacatcggtgactcctcagcggttctatgcagatctggaaaagcagaagttctgactagtcctcaccgtccatatgggagcaacaaaacttctcttaatgaaattagaagaatcagagaagcaggtggatggattagcaatggtagaatttgtggagacattgctgtatctcgtgcatttggggacatgcgattcaaaacgaagaagaatgagatgctgcaaaaaggagttcaggaaggaaaatggtcagcaaagttcatctctcgtgtacagctcaacaatgacttggttgtagcataccctgatatttatcaggtagctcttggctctgatgcagaatttatagtattagcaaccgatggcttatg

SEQ ID NO: 60 - DNA, 700 bp, Vigna unguiculata
gaggtgctacagcaactgccattttcatcggagatgatgagcttctaatttcacatatcggtgactcttctgcggttctatgcagatctggaaaagcagaagttctgaccagtcctcaccgtccatatgggagcaacaaaacttctcttgatgaaattagaagaataagagaagcaggtggatggattagcaatggtagaatttgtggagacattgctgtatctcgtgcatttggggacgtgcgattcaaaacgaagaagaatgagatgctgcaaaaaggagttcaggaaggaaaatggtcagcaaagttcatctctcgtgtacagctcaacaatgacttggtcgtagcataccctgatatttatcaggtagctcttggctcagatgcagaatttgtagtattagcaaccgatggcttatgggattacatgagcagctcagaagcagtttcttttgttagggatcaactacgaaaacatggaaacattcagctagcatgtgaatcgcttgcagaagccgctttggaccgacgtacacaggataatgtcagcatcataattgctgatttagggcgaacagactggcagaatgtgccactggagcgacaaaatactatagtcgagctggttcaagctcttgctaccattggaattgtgtcttttggaatctggttttcatcacagctttctctatagatgca

SEQ ID NO: 61 - DNA, 715 bp, Triphysaria pusilla
cgcaattagtgagggaagacgagtccggctcgacggctactactattttcctctgcgacgatgtattggttgtttctcacgttggtgattcgtgcgtggttctttcacgcgatggaaaagttgaagtcttgactgattctcatcggccctacggaaataacaaagcctctttacaagaaatcaggcgaattcgagaggctggtggatggattgtaaatggaaggatttgtggtgacatatctgtatcgcgtgcttttggagatatacgatttaaaactaagaaaaatgaaatggtggcgaaaggagtcgaggaaggccgatggccggcaaaatttgcttctcgcttaaaattgaaaggagacttggttactgcatgtccagatgtttttcaagtttctataggatctgatgccgaatttatagtattagcatctgatggtttgtgggactacataaacagctcggatactgttaaattcattagaaatcaccttcgacaatatggagatgttcagatggcttctgaagctcttgctcaaatggctttgaaccaaaattcacaagataacatcagcattattattgcagatctcggaagaacggattggaaaaaattgcctgtgcagcagcaaaatatcgccgttgagttggttcaagctctaaacactctcagtttagtatcacttggaatatggatatcgagtttgctttcaac

SEQ ID NO: 62 - DNA, 806 bp, Carthamus tinctorius
tggagacgatgctgttatagtcgcttcatcatcgtctaataacgaccttgatggtttccattttgctgctgttttcgatggctatgccggattctcttccgtcgacttcctcagggaggagctatacaaggagtgtgtgaaggctcttcaaggggggctattgttgagtaaaaaagtgaccgaaattgaaaaggcgttgcaagaggccttcatgaacgctgatgcaaaacttctgaactggcttgagatgagcgggaaggacgacgaatctggtgcaacggctactactatgtttgtaggaaatgatatgctattcatatcccatgttggtgactcatgtgcggtactttcggaatctggaaaaccggaagtgctgactaattctcacagaccctatggaagacacaaggtttctcttcaagaaattaaacgagtcagagaagcaggtggatggattgtggatggaagaatttgtggagatatctctatatcccgtgcctttggggacatacggtttaagacgaagaagaccgagatgctggaaaagggtgtcgaggaaggaagatggacccaaaaatttatatcacgcattcggttttctggagatttggtcattgctactcctggtatttatcatgtgacgacggggtctgatgcagagtttgtactgttagcaactgatggcttatgggattacattaacagttcagatgctgtcaactttgtcagaaaccaacttagggaacatggagatgttcaggctgcatctgaagctctagctcaaatggcactggaccaatattctc

SEQ ID NO: 63 - DNA, 801 bp, Citrus reticulata
  misc_feature (687)..(687): n is a, c, g, or t
ccacgcgtccggctactgctcggcgattgcaatagacgcgccgtcttcgttcagtggtgttgccggaatccgatgggggtctgtaagtttacagggcctacgtgaggaaatggaggatggtgctgttattcaatcggatggccttgatgggttctcttttgcagctgtttttgatggccatggtggagtctctactgtcaagtttcttagggatgagctatacaaagaatgtgttgcagctttacaaggaggattgctgttgagtggaaaagattttgatgccattaagaagggattacaggaggcttttgaaaatgctgatatgaaattgttgaattggcttgaaatgaatgcggaggaggatgaatctggtgcaaccgctactgttgtgttcattggaagggatattttgttcatctcacatattggtgactgttgtgtggttctatctcgtactggaaaagcagatgtattgactaatcctcatcggccttatggaagcagccaggtctcacttcaggaaattagaagaatcagagaagcgggcggatggatttccaatggtaggatatgtggtgacattgctgtatctcgtgcttttggtgacttccggttcaagaacaagaaaaatgagatgctgaagaaaggtgttaatgaaggaagatggtcagaaaaaatttgttctanaagtgcactttacaaagacctggttattgcatcaccagatatttttcaagtatctcttggggtcagatgcagaatttgtactgtttacctctgatggactatgggattacctaa

SEQ ID NO: 64 - DNA, 960 bp, Lactuca sativa
  misc_feature (818)..(818): n is a, c, g, or t
cgggcttttctgcaactcatcttctctcatccatggctttatggagtccacgcttgcaaagatttctactcaacaacaacgttagtaagatcgacttgaagaagactaatagttgtaatagcaataatcatataattatgagaaagtggagtagagaaggcagcccccggtgctgttcggcgattgcaatcgacgctccttcttctctctctgatggccttggctctgggatcagatggggttccgccaaattacaaggtgcccgcgaggagatggaagacgatgctgttatagtcggctcatcgcctcataacgagcttgatggtttctttttcgctgccgttttcgatggtcacgcgggtttctcttctgttaagttcctcagggaggagctatacaaggagtgtgtgaaggctctacaaggggggctattgttgagtaaaaaggacaccattggaatcgagaaagcactgcaggaggctttccagaatgctgatgccaaacttctaaactggcttgagatgaacggagaggacgatgaatctggtgcaacagccacaactatgttcctaggaaacaatatgctgttcatctctcatgtcggtgattcatgtgcggttctttcacaatctggaaaggcacaagaattgaccaattctcacagaccctatggaagaaacaaggtttcccttcaagagatcaagcgaatcagagaagcaggtggatggatttcggatggaagaatctgtggagatatttctgtatcccgtgcctttggtgacatgcggtttaaaacaaagaagaaagagatgctgganaagggagttgccgaaagcagatggtctgaaaaatttgtatcccgcatgcggttctttggggacttggttattgctactcctgatatttatcaagttacacttgggcctgaagcagaattttgtatggtagcgaccgatggct

SEQ ID NO: 65 - DNA, 693 bp, Gossypium hirsutum
  misc_feature (660)..(660): n is a, c, g, or t
gcgacgatgaatccggttcaaccgccacagtcatgttaattggaaatgaagtgctcttcatttctcatgttggtgattcttgtgtggttttgtcttgtgctggaaaagtacaagtcttgactgattctcatcgaccctatggcagcaacaaggcctctttgcaagaaattagaagaatcagagaagcaggtggatggattagcaatggtaggatttgtggtgacatagctgtatcccgtgcttttggcgacactcgatttaaaacgaagaagaacgaaatgctgaagaagggagttgaagaaaagagatggtctgaaaaatttatttctcgtgtagtgttcaatgatgacttggtcatagcatctccagacacttttaaagtagctcttggatcagatgcagaatttgtcctgttggcatctgatggtttatgggattacataaacagttcagacgcagttgcttttgttcgaaatcaattacgagaacatggagatgttcaggtagcatgtgatgtacttgcacaagccgctctggataaaggctcagaagacaatgtcagcattatcatagctgatttcgggcatactgaatggcagaaattgccagttgagcaacaaaattttctgtttgaatttggccaagctatagctacagtangggtcgtgtcacttggaatatggttgtcatctc

SEQ ID NO: 66 - DNA, 707 bp, Helianthus annuus
tttctcttctgtcaactttctcagggaggagctgtacaaggagtgtgtgaaggctctacaagggggccaattgttggataataaagactttattggaattaaaaatgccctgcaagaagcttttcacaacgctgacgccaaacttctaaactggcttgagatgaccagtgaggaggatggtgaatctggtgcaacagctactgctatcttcataggaaatgatatgctatatatctcacatgttggtgactcatgtgccgttctttcacaatctggaaacgcagaggaactcactaattctcacagaccctatggaagaaacaaggtttctcttcaagaaatcaaacgcattcgagaagcaggtggatggatttcggatggaagaatttgtggagacatctctgtatcacgtgcctttggtgacatacggtttaagaccaagaagaatgagatgctgaaaaagggagttgaagaaggaagatggtccgaaaagtttgcatcccgaatgaagttttttgaagacttggttattgctactcctgatatttatcaagttgcattggggcctgatgcggagtttgtattattagcaactgatggcttatgggattacattaagagctcggatgcagtcagctttatcagaaaccaactacgggaacacggagatgttcaggttgcatctgaagcgctagctcaaatggcct

SEQ ID NO: 67 - DNA, 759 bp, Glycine max
gaaggatgagctgtacaaggagtgtgttgaagctttacaaggtgggttactattagtcgagaaagatttcaaagccattaaaagggcattacaggaggcatttctcaaggctgacgcgaggttattaaaacggcttgaaatgaatggagaggaggatgaatcaggtgctacatcaactgccgtgttcattggagatgatgagctgctaatttcacatattggtgactcatcagcggttctatgtagatctggaaaagcagaagttctgactagtcctcaccgtccaattgggagcagcaagacttctcttcatgaaattagaagagtcagagaagcaggtggatggattaacaatggtagaatttgtggagacattgctgtatctcgtgcatttggggacgtgcgattcaaaacaaagaagaatgagatgttgcaaaaaggagttcaggaaggaagatggtcagcaaagttcatttctcgtgtacaactcaacaacgacttggttgttgcataccctgatatttatcaagtaactcttggctcagatgcggaatttgtagtattagcatccgatggcttatgggattacatgagcagctcagaagcagtttctttagtgagggatcaactacgaaaacacggaaacattcagcaagcatgtgaagcgcttgcagaagccgctttggaccgacgtacacaggataatgtcagcattataattgctgattttgggaggacagactggcagaatgcgccatt

SEQ ID NO: 68 - DNA, 827 bp, Avena barbata
gttggatggtggagcagtgctcaacaccaagaatcttgaagccatcacggaatcgatcaggcgtgcctttgccactgtggacgccaacctctccacctggcttgagaaaatggacaaagaagacgaatctggcgccacagccacggccttgtttctgaggaaagatgtccttgttgtctcgcacattggtgactcctgcttgcaggtcgtatcacgtggcggaagacctcaatctctgaccaactttcatcggccatacgggcacaacaaaacatcccttgaagaggtcaagcggattagagcagcaggtggatggattctcgatggacgcatatgtggggacatctctgtatctcgagcttttggagacataagatttaagacacggaagaatgagatgctggtaaaaggagttaaagaaggaagatggactgaaaaattcgtttcaagaataaaatttaaaggggatctaataatctcttcacctgatgtatctctggtggagcttggaccggatgttgaatttgttttggttgcaactgacggcctttgggattacattaaaagcactgaagctgtagcttttgtcagagatcaacttcgtcaacatggagatgtccagcgggcctgtgaggcgcttggggagaaagctctggatcaacggtctcaagataacattagcatagtcatagctgacttgggaaaggacaaactggaaaggagttgcccgtaccaaggccaaacgtgttcttggagctaagtcaagcagttgcaactgtaggggctgtgtcactaggaatttggatttcgtcacttcttaccctgca

SEQ ID NO: 69 - DNA, 792 bp, Panicum virgatum
gctggacggcggcgcggtgctcagcaccaagaatctcgaggccatcacggcctccatccagcgtgccttcgccgcagtcgacgcgaagctctccacctggcttgagcaaatggacaaggacgacgactctggcgccacggctactgccatgtttctcaggaacgatgtccttgttgtttcgcacattggggactcttgcctgatgatatcgcgcggtggaagacctgaagctttgacgagctctcatcgaccgtatgggaacaacaaaacatcacttgaagaggtcaaaaggatcagggcagcaggtggatggattgttgatgggcgcatatgtggagacatttctgtatctcgtgcttttggggacataagatttaagacacgaaaggatgagatgctagtgaaaggagttaaagaaggaaggtggactgaaaagttcatctcacgaataaaatttaaagaggatataataatctcgtcacctgatgtatctctggttgagcttggaccggatgtggaatttcttatactagcaactgatggtctttgggattacatgaaaagctctgaagctgtagcatttgtccgagatcaactacgtcaacatggtgatgtccagttggcctgtgaggcacttggtcagaaagctctggatgaacggtcacaagacaatattagcatagtcatagctgacttggggaggacaaactggaaggcgttgcccgtggaaaggccaaacttgttcttgggaactaagtcaagcagttgtgacttgtaggggttgtcgcagta

SEQ ID NO: 70 - DNA, 743 bp, Artemisia annua
atgctaatgcaaccacatagataagccatggaagtaagccgttatacacttacagtaatagaatcttaatagactatatacagagattgcaaggttcgttctcaaaatgaagcattagatgacatccatattccaattgtcacaataccgatggttgcaaaggcttggatgagttcaaagacaaagttttgctgttgtataggtaaatttcgccaatctgtccgccccaaatcagcaatgacaatggttacattatcttgagaatactggtccaaagccatttgagctagagcttcagatgcaacctgaacatctccatgttctctaagttggtttctgacaaagttgactgcttccgagcttttaatgtagtcccataagccatcagttgctaacaatacaaactcggcatcaggaccgagagtaacatgataaatatcaggagtagcaataaccaagtctccaacaaactttatgcgagagacaaatttttcagaccatcttccttctgcaagtcccttttccagcatctcattcttcttcgtcttaaacctcgtgtcaccaaaggcacgagatacagatatatctccacagattcttccatccgaaatccatccacctgcttctctgattcgcttgatttcttgaagagaagccttgcttcttccataaggtctgtgagaatcggttagttcttctgcttttccagatagtgaaagaaccgcgcttgagtcaccaatatg

SEQ ID NO: 71 - DNA, 703 bp, Allium cepa
gtcagagatggaagatgatgttgtaatgagacttgacgggcttgatggatttgcctttgcagccgtttttgatggccatgctgggttctcttctgttgaatttctcagggacgagttatacaaagagtgcgtagctgcattgcaaggtggttcattgctgactagtaagaactttaatgctataaaagatggaattgcaaaggcttttcgaacagttgatattaaactgtcatcttggcttgaacaagtgggaaaggacgctgactctggtgcgacagctacagccatgttcctgggaaaggatgtattgataatttcacatgttggtgattcgagtgtggtcatatcaagaaatgggaaagctgaaatcttaacaggtccacatagaccatatggcaacaacaaagtttctcttgaagaagtcaagcggataagagcagctggtggatggatcattgatggaagaatttgtggtgatatctctgtgtcccgtgctttcggggatatacgattcaaggcaaagaagaatgaaatgctaagaaaaggagttgaagaggggagatggacagaaaaatttgtctccagaatacgattcaaaggtgacttggtaacttcttcccctgatgtctttcaacttgaattgggatctgatgtggaatttgtattgcttgcatctgatgggttatgggactatattaggagc

SEQ ID NO: 72 - DNA, 607 bp, Arachis hypogaea
tggttctatctagagctggaaaagcagaaatgcttactagtcctcacagaccatatgggaggaacaaggcttctcttcaagaaatcaaaagaatcagagaagcaggtggatggattgtgaatggtagaatttgtggagacattgctgtatctcgtgcattcggggacttacgatacaaaacaaagaagaatgagatgctacaaaaaggagttcaagaaggacgatggtcagaaaagtttatctctcgggtacggctcaacaatgatttagttgttgcataccctgatatttatcaagtagatcttggctcagatgctgagtttatagtattagcgtcagatggcttatgggattatatgagcagctcagatgcagtttcctttgtgagggaacagttacaaaagcatggaaacattcaggtagcctgtgaagcacttgcacaagcagctatggaccgtagaacacaggataatgtgagcattataattgctgatttagggaagacagattgggagaatgtggcagtgcagcaacaaaatatggtattggagctggttcaagcccttgctaccattggtattgtgtccattggaatttggttttcgtc

(19) United States
(12) Patent Application Publication
(10) Pub. No.: US 2012/0272400 A1
(43) Pub. Date: Oct. 25, 2012

(54) Photosynthesis Rate

(76) Inventors: Dario Leister, Munich (DE); Mathias Pribil, Munich (DE)

(21) Appl. No.: 13/504,062

(22) PCT Filed: Oct. 29, 2010
(86) PCT No.: PCT/EP10/06632

§ 371 Date: Jul. 9, 2012

(30) Foreign Application Priority Data
Oct. 29, 2009 (EP) 09013639.1

(51) Int. Cl. C12N 15/82; A01H 5/00; A01P 21/00; C12N 15/01; A01H 1/06; A01N 59/10; C12N 5/04; C12N 5/10

(52) U.S. Cl. 800/276; 435/410; 800/298; 435/411; 435/412; 435/417; 435/414; 435/415; 435/416; 800/317.4; 800/320.1; 800/320.3; 800/320; 800/320.2; 800/307; 800/310; 800/317; 800/305; 800/306; 800/317.2; 800/309; 800/313; 800/316; 800/317.3; 800/314; 800/322; 800/312; 435/445; 435/446; 435/468; 800/278; 800/286; 504/116.1; 800/290

(57) ABSTRACT

The invention relates to methods of increasing the photosynthesis rate of a plant cell and/or the production of biomass in a plant, as well as to plant cells and plants with increased photosynthesis rate/production of biomass. The present invention also relates to a phosphatase of the light-harvesting complex of photosystem II, to nucleic acids coding for such phosphatase, and to mutants of such nucleic acids. Moreover, the present invention relates to plant cells and plants, wherein the activity of said phosphatase is inhibited; in particular the present invention relates to plant cells and plants comprising the mutant nucleic acids.

The invention relates to methods of increasing the photosynthesis rate of a plant cell and/or the production of biomass in a plant, as well as to plant cells and plants with increased photosynthesis rate/production of biomass. The present invention also relates to a phosphatase of the light-harvesting complex of photosystem II, to nucleic acids coding for such phosphatase, and to mutants of such nucleic acids. Moreover, the present invention relates to plant cells and plants, wherein the activity of said phosphatase is inhibited; in particular the present invention relates to plant cells and plants comprising the mutant nucleic acids.

A growing world population and a simultaneous reduction in farmland due to various factors (e.g. salinization) lead to aggrevating problems of world-wide food shortage. Similarly, ongoing climate changes require a continuous development of improved varieties of agricultural crops that are adapted to the new climate conditions (for example by increased tolerance to salt).

Traditionally, such improvements have been achieved by breeding, i.e. by introduction of the desired traits by crossbreeding and selection. In the wake of time and cost reduction, approaches based on genetic engineering are of increasing importance. This involves the targeted alteration of individual genes or proteins or even complete biosynthetic pathways, in order to obtain the desired improved characteristics. Frequently, an enhanced growth rate and thus an increase in the production of biomass is a designated goal in various areas of agricultural production, especially in the holistic utilization of plants (e.g. tree/wood production; sugarcane/sugar production, PHB (polyhydroxybutyrate, a bioplastic); vegetable plants/food production, plantibodies, oral vaccines; etc.). Indirectly, an enhanced growth rate can also lead to an increase in the production, e.g. in the case of grains. “Growth rate” and “biomass production” are controlled by multiple genes, which are very hard to identify for the purpose of genetic engineering.

A significant part of agricultural food production nowadays happens in large-scale greenhouses under controlled growth conditions (light, temperature, humidity). Due to changes in climate that may be imminent, intensified use of transgenic agricultural crops coupled with a still low acceptance of such plants in the public, an increase in the area of closed off greenhouse facilities is expected. An increased production of biomass under conditions of low light (reduction of energy cost) is therefore likely to become more and more important.

Of critical importance for the efficient production of biomass through a plant is the availability of energy carriers (ATP/reduction equivalents) and carbohydrates. These compounds are the products of photosynthesis, which therefore plays a central role in biomass production.

Photosynthesis is a process by which solar energy is converted into chemical bond energy. The overall reaction of photosynthesis is the light-driven conversion of carbon dioxide and water to glucose and oxygen:

6CO₂+6H₂O→C₆H₁₂O₆+6O₂

The glucose is then used as a source of chemical bond energy as well as for the formation of various compounds that are essential for plant cell metabolism, and thus for plant survival and growth.

Photosynthesis is observed in plants as well as some protists, bacteria, and blue-green algae. The site of photosynthesis in plants is the chloroplast, a subcellular organelle that is found mainly in cells of plant leaves. Chloroplasts are cytoplasmic organelles surrounded by a double membrane. Enclosed by the inner membrane is the stroma, a soluble phase rich in enzymes. The stroma, in turn, contains a third membranous compartment, the thylakoids. These are flattened, sac-like membrane structures often forming stacks called grana.

Photosynthesis comprises two stages called the light reactions and the dark reactions. The light reactions require the presence of light, while the dark reactions do not depend on direct light exposure (nevertheless in most plants the dark reactions happen during the day). The thylakoid membranes are the site of the light reactions, whereas the dark reactions take place in the stroma of the chloroplast. In the light reactions, sunlight is absorbed and drives an electron transport chain that results in the formation of the energy carriers ATP and NADPH, forming O₂ as a by-product. In the dark reactions, a reaction driven by NADPH and ATP reduces CO₂ to glucose.

As the first step of the light reactions, energy from sunlight is absorbed by the green organic pigment chlorophyll in the thylakoid membrane and energizes an electron. Large multiprotein complexes called photosystems then allow, in collaboration with other components (such as the cytochrome b₆/f complex and the mobile components plastoquinone, plastocyanin, and ferredoxin), the conversion of the solar energy captured in excited chlorophyll molecules into chemical bond energy via an electron transport chain.

In general, photosystems are made up of two components: (1) a photochemical reaction center that consists of a complex of proteins and chlorophyll molecules and allows solar energy to be converted into chemical energy and (2) an antenna complex, which consists of a number of distinct membrane protein complexes (the light-harvesting complexes) that bind chlorophyll molecules and other pigments, which capture light energy and transfer it to the reaction center, resulting in excitation of the photosystem. In green plants, there are two photosystems called photosystem I and II (PSI and PSII).

Owing to their sessile life style, plants have to cope with environmental changes in their habitats, such as fluctuations in the incident light. Changes in light quantity or quality (i.e. spectral composition) result in imbalanced excitation of the two photosystems and decrease the efficiency of the photosynthetic light reactions. Plants can counteract such excitation imbalances within minutes by a mechanism called ‘state transitions’, which depends on the reversible association of the mobile pool of major light-harvesting (LHCII) proteins with photosystem II (state 1) or photosystem I (PSI) (state 2). In detail, the phosphorylation of LHCII, stimulated in low white light or by light of wavelengths specifically exciting PSII (red light), causes association of LHCII with PSI (state 2), thus directing additional excitation energy to PSI. Conditions like darkness or light of wavelengths specifically exciting PSI (far-red light), as well as high intensities of white light, stimulate LHCII dephosphorylation and its migration to PSII (state 1), thus re-directing excitation energy to PSII.

The protein kinase responsible for phosphorylating LHCII is membrane-bound and activated upon reduction of the cytochrome b₆/f (Cyt b₆/f) complex via the plastoquinone (PQ) pool under state 2-promoting light conditions (low white light or red light). PQ oxidizing conditions induced by state 1-promoting light conditions (dark or far-red light) inactivate the LHCII kinase and result in association of LHCII with PSII (state 1). The LHCII kinase activity, however, is also inactivated under high white light conditions, when the stromal reduction state is very high. In vitro and, more recently, in vivo studies suggest that suppression of LHCII kinase activity might be mediated by reduced thioredoxin. In C. reinhardtii and A. thaliana, the orthologous thylakoid protein kinases Stt7 and STN7, respectively, are required for LHCII phosphorylation and state transitions. Co-immunoprecipitation assays showed that the Stt7 kinase interacts with Cyt b₆/f, PSI and LHCII, suggesting that Stt7 (and STN7 in Arabidopsis) directly phosphorylates LHCII, rather than being part of a Stt7/STN7-dependent phosphorylation cascade.

Under PQ oxidizing conditions when the LHCII kinase becomes inactivated, pLHCII is dephosphorylated by the action of a protein phosphatase, thus allowing the association of the mobile fraction of LHCII with PSII (state 1). Despite various attempts, however, nobody has been able to identify the responsible phosphatase.

To meet the increasing world food demands as well as for economic reasons, enhancing the rate of plant growth and hence of biomass production is becoming more and more important. To achieve this goal, it is of central importance to generate plant varieties with improved growth rate under suboptimal conditions, such as the conditions of low light found in some greenhouses.

Enhancing photosynthetic activity by fixing the photosynthetic machinery in one state would be an ideal approach to optimally adapt plants to specific conditions of light. For example, by shifting the balance of the phosphorylation state of LHCII completely to the side of the phosphorylated form (state 2), the photosynthesis rate of a plant at low light conditions could be optimized. One way to accomplish this would be to inactivate the phosphatase responsible for LHCII phosphorylation. However, so far there has been little success to increase the efficiency of photosynthesis by targeted gene technological approaches. In particular, as pointed out before nobody has succeeded in identifying the LHCII protein phosphatase(s) or discovering ways how to inhibit its activity.

One object of the present invention was therefore to provide a method for increasing the photosynthetic activity of a plant cell.

A further object of the present invention was to provide a plant in which the photosynthetic activity especially under low light conditions is increased.

These objects of the present invention are solved by a plant cell or plant, wherein the activity of a phosphatase of the light harvesting complex of photosystem II has been reduced or inhibited.

In one embodiment, said phosphatase has an N-terminal chloroplast transit peptide (cTP), a C-terminal transmembrane domain and a protein phosphatase 2C signature.

In one embodiment, the activity of said phosphatase of the light harvesting complex of photosystem II has been reduced or inhibited by one of the following:
(i) producing a mutant nucleic acid coding for a non-functional version of said phosphatase, through insertion, deletion or mutation of at least one nucleotide in the nucleic acid wildtype coding sequence of said phosphatase, and introducing said mutant nucleic acid into a plant cell,
(ii) producing a mutant nucleic acid coding for a version of said phosphatase having reduced expression, through insertion, deletion, mutation of at least one nucleotide at a position 1-500 nucleotides, preferably 1-100 nucleotides upstream of the nucleic acid wildtype coding sequence of said phosphatase, and introducing said mutant nucleic acid into a plant cell,
(iii) producing a mutant nucleic acid by placing the nucleic acid coding for said phosphatase under the control of a regulatory sequence that switches off the expression of said nucleic acid coding for said phosphatase, and introducing said mutant nucleic acid into a plant cell,
(iv) producing, within a plant cell, a mutant nucleic acid coding for a non-functional version of said phosphatase or a version of said phosphatase having reduced expression, by exposing said plant cell to a mutagenic substance, such as ethyl methane sulfonate, or by exposing said plant cell to mutagenic radiation, such as UV-radiation or X-rays, and identifying the resultant mutant nucleic acid based on the recognition of mismatch hybridizations,
(v) introducing siRNA directed at the nucleic acid coding for said phosphatase, into a plant cell, or
(vi) blocking the activity of said phosphatase within a plant cell at the protein level by exposing said plant cell to a phosphatase inhibitor, such as NaF.

In one embodiment, said plant cell or plant is selected from monocotyledons or dicotyledons or eudicotyledons.

In one embodiment, said plant cell or plant is from a plant useful for nutrition, in particular human nutrition or animal nutrition.

In one embodiment, said plant cell or plant is from a species selected from flowering plants.

In one embodiment, said plant cell or plant is from a species selected from greenhouse plants.

In one embodiment, said plant cell or plant is from a plant useful for the production of biomass.

In one embodiment, said plant cell or plant is from a species selected from tomato (Lycopersicon esculentum), corn (Zea mays), wheat (Triticum aestivum), Sorghum, rice (Oryza sativa), barley (Hordeum vulgare), cucumber (Cucumis sativus), courgette or zucchini (Cucurbita pepo), squash or gourd or pumpkin (Cucurbita pepo, Cucurbita mixta, Cucurbita maxima, Cucurbita moschata), eggplant or aubergine (Solanum melongena), lettuce (Lactuca sativa), spinach (Spinacia oleracea), cabbage (Brassica oleracea), chicory (Cichorium intybus), corn salad (Valerianella locusta), broccoli (Brassica oleracea), cauliflower (Brassica oleracea), common grape vine (Vitis vinifera), sugar beet (Beta vulgaris), beetroot (Beta vulgaris), thale cress (Arabidopsis thaliana), castor oil plant (Ricinus communis), Western Balsam Poplar (Populus Trichocarpa), Sitka spruce (Picea sitchensis); herb species such as from basil (Ocimum basilicum), thyme (Thymus subsp.), sage (such as Salvia species), chives (Allium schoenoprasum), parsley (Petroselinum crispum, Petroselinum neapolitanum) etc.; berry species such as from strawberries (Fragaria×ananassa), Ribes species, Vaccinium species, Rubus species etc.; potato (Solanum tuberosum), carrot (Daucus carota), leek (Allium ampeloprasum), onion (Allium cepa), Piper species or Capsicum species such as from sweet pepper or hot pepper etc., melon species such as from watermelon (Citrullus lanatus) or muskmelon (Cucumis melo), okra (Abelmoschus esculentus), beans such as Vicia species, Vigna species, such as Vigna unguiculata, Phaseolus species, such as Phaseolus vulgaris, climbing beans, dwarf beans, broad beans, asparagus (Asparagus officinalis), artichoke (Cynara cardunculus), celeriac (Apium graveolens), peas (Pisum sativum); Citrus species or Actinidia species, Chinese cabbage (Brassica rapa), rapeseed (Brassica napus), tobacco (Nicotiana tabacum), radish (Raphanus sativus), orange (citrus sinensis), wild radish (Raphanus raphanistrum), Columbine species (Aquilegia sp.), clementine (Citrus clementina), owl's clover species (Triphysaria sp., such as Triphysaria pusilla), safflower (Carthamus tinctorius), mandarin (Citrus reticulata), upland cotton (Gossypium hirsutum), sunflower (Helianthus annuus), soybean (Glycine max), oat species such as Avena sativa or Avena barbata, switchgrass (Panicum virgatum), annual wormwood (Artemisia annua), and nut species such as peanut (Arachis hypogaea).

In one embodiment, said phosphatase is a phosphatase of the light harvesting complex of photosystem II and has an amino acid sequence which is
a) SEQ ID NO:1,
b) at least 50%, preferably at least 60%, more preferably at least 70%, even more preferably at least 80%, even more preferably at least 90%, and most preferably at least 99% identical to SEQ ID NO:1, or
c) a homologue to SEQ ID NO:1 from a plant species other than Arabidopsis thaliana, said homologue having an N-terminal chloroplast transit peptide (cTP), a C-terminal transmembrane domain and a protein phosphatase 2C signature.

In one embodiment, said homologue has an amino acid sequence selected from SEQ ID NO:2-11 and 23-47.

In one embodiment, said plant cell or plant has an increased photosynthesis rate or increased production of biomass in comparison with a plant cell or plant, wherein said phosphatase has not been inhibited.

The objects of the present invention are also solved by a method of increasing the photosynthesis rate and/or the production of biomass in a plant cell or plant, said method comprising:
reducing or inhibiting, in said plant cell or plant, the activity of a phosphatase of the light harvesting complex of photosystem II.

In one embodiment, said phosphatase has an amino acid sequence which is
a) SEQ ID NO 1:,
b) at least 50%, preferably at least 60%, more preferably at least 70%, even more preferably at least 80%, even more preferably at least 90%, and most preferably at least 99% identical to SEQ ID NO: 1, or
c) a homologue to SEQ ID NO: 1 from a plant species other than Arabidopsis thaliana, said homologue having an N-terminal chloroplast transit peptide (cTP), a C-terminal transmembrane domain and a protein phosphatase 2C signature.

In one embodiment, said homologue has an amino acid sequence being selected from SEQ ID NO:2-11, and 23-47.

In one embodiment said reducing or inhibiting the activity of said phosphatase occurs by one of the following:
(i) producing a mutant nucleic acid coding for a non-functional version of said phosphatase, through insertion, deletion or mutation of at least one nucleotide in the nucleic acid wildtype coding sequence of said phosphatase, and introducing said mutant nucleic acid into a plant cell,
(ii) producing a mutant nucleic acid coding for a version of said phosphatase having reduced expression, through insertion, deletion, mutation of at least one nucleotide at a position 1-500 nucleotides, preferably 1-100 nucleotides, upstream of the nucleic acid wildtype coding sequence of said phosphatase, and introducing said mutant nucleic acid into a plant cell,
(iii) producing a mutant nucleic acid by placing the nucleic acid coding for said phosphatase under the control of a regulatory sequence that switches off the expression of said nucleic acid coding for said phosphatase, and introducing said mutant nucleic acid into a plant cell,
(iv) producing, within a plant cell, a mutant nucleic acid coding for a non-functional version of said phosphatase or a version of said phosphatase having reduced expression, by exposing said plant cell to a mutagenic substance, such as ethyl methane sulfonate, or by exposing said plant cell to mutagenic radiation, such as UV-radiation or X-rays, and identifying the resultant mutant nucleic acid based on the recognition of mismatch hybridizations,
(v) introducing siRNA directed at the nucleic acid coding for said phosphatase, into a plant cell, or
(vi) blocking the activity of said phosphatase within a plant cell at the protein level by exposing said plant cell to a phosphatase inhibitor, such as NaF.

Exposing plant cells to mutagenic substances, such as ethyl methane sulfonate (EMS), or exposure to mutagenic radiation, leads to point mutations in the DNA sequence of TAP38 and therewith to its ‘inactivation’ without the introduction of transgenic material (as in the case of T-DNA insertions). These point mutations can be identified using, as an example, the TILLING-method (“targetting induced local lesions in genomes”), which is based on the recognition of mismatch-hybridisation via HPLC. Plants generated via such mutagenesis, e.g. EMS mutagenesis are by definition not genetically engineered and can be handled outside of ‘S1’-facilities.

In one embodiment increasing the photosynthesis rate and/or the production of biomass occurs under light exposure of said plant cell or plant to photosynthetic active radiation (PAR) in the range 50-200 μmol m⁻² s⁻¹, preferably 80-100 μmol m⁻²s⁻¹.

In one embodiment the nucleic acid coding for said phosphatase has a nucleic acid sequence selected from SEQ ID NO: 12-22, and 48-72.

In one embodiment the siRNA consists of 15 to 21, or 21 to 50, or 50 to 100, or 100 to 150, or 150 to 200 contiguous nucleotides of one of SEQ ID NO: 12-22 and 48-72, alone or in combination with its respective complementary counterpart strand.

In one embodiment said mutant nucleic acid is a mutant nucleic acid the expression of which in a plant cell results in at least 80% less transcripts of said phosphatase, said mutant nucleic acid having an insertion, deletion or mutation of at least one nucleotide in the nucleic acid wildtype coding sequence of said phosphatase, or said mutant nucleic acid having an insertion, deletion or mutation of at least one nucleotide upstream of the nucleic acid wildtype coding sequence of said phosphatase.

In one embodiment said mutant nucleic acid has a nucleic acid sequence selected from SEQ ID NO: 12-22 and 48-72, into which or upstream of which a stretch of nucleotides has been inserted. Such stretch of nucleotides may, in the simplest case, consist of one nucleotide; it may, however, also consist of 10 nucleotides to 30000 nucleotides. This may, e.g., be achieved through the use of T-DNA which T-DNA may be derived from a number of bacteria, e.g. Agrobacterium tumefaciens. If the insertion is upstream of such coding sequence, it is preferably 1-500 nucleotides, more preferably 1-100 nucleotides, upstream of the start codon.

As an example, two mutant lines in A. thaliana that can be used in accordance with embodiments of the present invention carry T-DNA insertions 49 nucleotides (first mutant: tap38-1) and 77 nucleotides (second mutant: tap38-2) upstream of the Start-codon. A third T-DNA insertion line (third mutant: tap38-3), also in accordance with the present invention, carries an insertion in the 4^{th} exon. The exact insertion-sites are highlighted in the following wildtype full length cDNA and were confirmed by sequencing. The entire length of T-DNA at this gene locus was not sequenced. The nomenclature of the respective T-DNA lines as listed in databases is the following:

| tap38-1(SAIL_514_C03); tap38-2 (SALK_025713); tap38-3 (GABI_232H12); | |
|---|---|
| 1 | AGATATTTTT TTACGAAAAT CCA-(tap38-2)-CGGCCAC TAACCGTAAA GGACACTCTT |
| 51 | T-(tap38-1)-TGATTTTTC TCTTCTTCTA CTTAACTCGC TTTGCCTGAC TCACTGAGTC |
| 101 | ATGGCGCTTC TGAGGCCGCA TCTTCATCGC TTTCACTCCA ACACTCTCCG |
| 151 | CCACTCAGCC TATCCTTCCG CCGACGCTGG CGGTGGCCTT GTCGTCTATC |
| 201 | CCACGTATGG TCGTCACCGT TGCTCCGCGA TTGCGATCGA CGCTCCGTCG |
| 251 | TCTCTGACTG GCGTGACGCC GATTCGTTGG GGTTACACGA GTGTTCAAGG |
| 301 | ATTCCGAGAT GAGATGGAAG ACGACATCGT AATCCGTTCT GATGCTGTCG |
| 351 | ATAGCTTCTC CTACGCAGCG GTCTTCGACG GCCACGCTGG GTCGTCCTCT |
| 401 | GTCAAATTCC TCAGGGAAGA GCTGTATAAA GAGTGTGTTG GTGCATTGCA |
| 451 | AGCTGGATCG TTGTTGAATG GAGGCGATTT CGCAGCAATC AAGGAAGCTT |
| 501 | TGATTAAGGC CTTTGAAAGC GTTGACCGCA ATTTGCTCAA ATGGCTAGAA |
| 551 | GCTAACGGTG ACGAAGAAGA TGAATCAGGT TCAACAGCCA CTGTAATGAT |
| 601 | CATCAGAAAT GATGTTTCCT TCATTGCTCA TATTGGTGAT TCGTGTGCAG |
| 651 | TCTTATCTCG ATCTGGGCAA ATCGAGGAAT TGACTGAT-(tap38-3)-TA TCACCGTCCA |
| 701 | TATGGAAGCA GTAGAGCAGC TATTCAGGAA GTGAAAAGGG TCAAAGAAGC |
| 751 | AGGTGGATGG ATTGTCAATG GAAGGATTTG TGGAGACATA GCTGTATCTC |
| 801 | GTGCTTTTGG TGACATCCGG TTTAAGACAA AGAAGAATGA TATGTTAAAG |
| 851 | AAGGGTGTTG ATGAAGGAAG ATGGTCCGAG AAGTTTGTTT CCAGAATCGA |
| 901 | GTTTAAAGGG GACATGGTTG TGGCGACTCC AGATATCTTC CAAGTACCTC |
| 951 | TGACATCTGA TGTGGAATTC ATTATTTTAG CTTCTGATGG ATTATGGGAC |
| 1001 | TACATGAAGA GCTCAGATGT GGTTAGTTAT GTTAGGGATC AGCTCCGGAA |
| 1051 | ACATGGGAAT GTGCAGCTTG CTTGTGAATC TCTTGCGCAA GTAGCTTTGG |
| 1101 | ACCGGCGATC TCAGGACAAC ATTAGCATCA TTATTGCTGA TCTAGGAAGA |
| 1151 | ACAGAGTGGA AGAATCTCCC CGCGCAGCGA CAAAACGTAG TGGTTGAACT |
| 1201 | GGTTCAGGCT GCAACGACTA TCGGTTTAGT TACTGTTGGC ATATGGATGT |
| 1251 | CTTCACATCT ATCTTAAGTG AATTGAACAT TCTATCTCTT TCGTTAGTGT |
| 1301 | ATATAAAACC TGTAACTATG TCTAACACTC TAGTCTTCCC ATAGAAATGG |
| 1351 | AGATATGAAG ATACCTCACA AACAGAACCA GCTTTATATT ATTATATATC |
| 1401 | CCACTTTCAC AAGCGTTG |

The objects of the present invention are also solved by a plant or plant cell that has been treated with the method in accordance with the present invention.

In one embodiment, said plant cell or plant is selected from monocotyledons or dicotyledons or eudicotyledons. In one embodiment, said plant cell or plant is selected from a plant useful for nutrition, in particular human nutrition or animal nutrition, or from a greenhouse plant or from a plant useful for the production of biomass. Merely exemplary embodiments include plant species as mentioned above.

The objects of the present invention are also solved by a nucleic acid having a nucleic acid sequence encoding a phosphatase of the light-harvesting complex of photosystem II, said phosphatase having an amino acid sequence which is
(a) SEQ ID NO: 1,
(b) at least 50%, preferably at least 60%, more preferably at least 70%, even more preferably at least 80%, even more preferably at least 90%, and most preferably at least 99% identical to SEQ ID NO: 1, or
(c) a homologue to SEQ ID NO: 1 from a plant species other than Arabidopsis thaliana, said homologue having an N-terminal chloroplast transit peptide (cTP), a C-terminal transmembrane domain and a protein phosphatase 2C signature.

In one embodiment, said homologue has an amino acid sequence being selected from SEQ ID NO: 2-11 and 23-47.

In one embodiment the nucleic acid according to the present invention has a nucleic acid sequence selected from SEQ ID NO: 12-22 and 48-72.

The objects of the present invention are also solved by a nucleic acid consisting of 15 to 21, or 21 to 50, or 50 to 100, or 100 to 150, or 150 to 200 contiguous nucleotides, preferably ribonucleotides, of the nucleic acid according to the present invention, alone or in combination with its complementary counterpart strand; in one embodiment, said nucleic acid consists of 15 to 21, or 21 to 50, or 50 to 100, or 100 to 150, or 150 to 200 contiguous nucleotides, preferably ribonucleotides, of one of SEQ ID NO: 12-22 and 48-72, alone or in combination with its respective complementary counterpart strand.

The objects of the present invention are also solved by a phosphatase of the light-harvesting complex of photosystem II, encoded by the nucleic acid according to the present invention.

The objects of the present invention are also solved by a mutant nucleic acid of the nucleic acid according to the present invention, the expression of which mutant nucleic acid in a plant cell results in at least 80% less transcripts of phosphatase of the light-harvesting complex of photosystem II, said mutant nucleic acid having an insertion, deletion or mutation of at least one nucleotide in the nucleic acid sequence as defined above, or said mutant nucleic acid having an insertion, deletion or mutation of at least one nucleotide upstream of the nucleic acid sequence as defined above.

In one embodiment the mutant nucleic acid according to the present invention has a nucleic acid sequence selected from SEQ ID NO: 12-22 and 48-72, into which or upstream of which a stretch of nucleotides has been inserted. Such stretch of nucleotides may, in the simplest case, consist of one nucleotide; it may, however, also consist of 10 nucleotides to 30000 nucleotides. This may, e.g. be achieved through the use of T-DNA, which T-DNA may be derived from a number of bacteria, e.g. Agrobacterium tumefaciens. If the insertion is upstream of such coding sequence, it is preferably 1-500 nucleotides, more preferably 1-100 nucleotides, upstream of the start codon.

The objects of the present invention are also solved by a plant cell comprising a nucleic acid as defined above or by a plant comprising one or several plant cells according to the present invention, wherein the plant cell and/or the plant are as defined above.

The inventors have recently identified a protein, namely a phosphatase of the light harvesting complex of photosystem II that is required for LHCII dephosphorylation and state transitions. This phosphatase is the long sought-after state transition phosphatase that dephosphorylates LHCII.

In plants without this phosphatase or having reduced or inhibited activity of such phosphatase, LHCII is constitutively hyper-phosphorylated, and enhanced thylakoid electron flow is observed, indicative of an increased rate of photosynthesis. The increased photosynthesis, in turn, results in more rapid growth, in particular under low light regimes.

As used herein, the term “low-light conditions” refers to conditions with light exposure in the range 50-200 μmol m⁻² s⁻¹ μmol m⁻² s⁻¹ photosynthetic active radiation (PAR), preferably 80-100 μmol m⁻²s⁻¹ PAR.

As used herein, the term “photosynthetic active radiation”, also abbreviated as “PAR”, is meant to refer to the spectral range that photosynthetic organisms are able to use in the process of photosynthesis. Preferably such spectral range is in the range of from 400 nm to 700 nm.

A photosynthesis rate that is “increased” in accordance with the present invention is with reference to the photosynthesis rate of a plant cell/plant that has not been treated or selected in accordance with the present invention. Hence, as an example, the point of reference may, for example be a wild type plant cell/plant or a plant cell/plant that has not been treated with the appropriate siRNA. There are numerous ways to measure the photosynthesis rate in accordance with the present invention, and these measurements are known to a person skilled in the art. For example, such measurements include, for example, and without being limited thereto, the determination of the fraction of Q_{A} present in the reduced state and measurements of the maximum quantum yield (F_{V}/F_{M}) and of the effective quantum yield (Φ_{II}) of photosystem II. Such measurements are known to a person skilled in the art and can be performed without difficulties.

The inventors have also found that a plant cell and/or plant that has been treated in accordance with the method according to the present invention has an increased biomass. Hence, a plant cell or plant treated in accordance with the present invention has an increased biomass, with reference to a plant cell/plant that has not been treated by a method according to the present invention.

Sometimes herein reference is made to a sequence a which is “at least x % identical” to a sequence b. This is meant to refer to a scenario, wherein at least x % of the residues (nucleotides, amino acids) of sequence b are also present in sequence a in corresponding positions.

Moreover, sometimes reference is made herein to a “homologue” to a sequence c. This is meant to refer to a nucleic acid or protein that is from another species than sequence c and that has the same physiological function in said other species. More specifically, a homologue to the phosphatase of the light-harvesting complex of photosystem II of Arabidopsis thaliana (SEQ ID NO:1) is also a phosphatase of the light-harvesting complex of photosystem II, but of another species. Such homologue is typically characterized by the presence of an N-terminal chloroplast transit peptide (cTP), a C-terminal transmembrane domain and a protein phosphatase 2C signature. Examples of such phoshphatase homologues are the corresponding phosphatases from Arabidopsis thaliana, Vitis vinifera, Oryza sativa, Hordeum vulgare, Lycopersicon esculentum, Zea mays, Triticum aestivum, Sorghum, Populus trichocarpa, Ricinus communis, Picea sitchensis, Cucumis sativus, Vigna species, such as Vigna unguiculata, Phaseolus species, such as Phaseolus vulgaris, asparagus (Asparagus officinalis), artichoke (Cynara cardunculus), celeriac (Apium graveolens), peas (Pisum sativum); Citrus species or Actinidia species, lettuce (Lactuca sativa), Chinese cabbage (Brassica rapa), rapeseed (Brassica napus), tobacco (Nicotiana tabacum), radish (Raphanus sativus), orange (citrus sinensis), wild radish (Raphanus raphanistrum), Columbine species (Aquilegia sp.), clementine (Citrus clementina), owl's clover species (Triphysaria sp., such as Triphysaria pusilla), safflower (Carthamus tinctorius), mandarin (Citrus reticulata), upland cotton (Gossypium hirsutum), sunflower (Helianthus annuus), soybean (Glycine max), oat species such as Avena sativa or Avena barbata, switchgrass (Panicum virgatum), annual wormwood (Artemisia annua), and nut species such as peanut (Arachis hypogaea). It should be noted that, due to the highly conserved character of the phosphatase sequences and of the mechanism of LHCII phosphorylation/dephosphorylation, experimental results obtained with Arabidopsis thaliana can also be extrapolated to other species. Arabidopsis thaliana is a system which works well as a photosynthetic model system for other plant species, such as flowering plant species.

Hammer et al. ((1995), Photosynth Res 106, 33-46) have shown that the regions of LHCII that are reversibly phosphorylated are highly conserved in their sequence between species. Wollman ((2001), EMBO J, 20, 3623-3630) states that state transitions are a mechanism that occurs generally in green plants and algae. From these data it can be inferred that experimental findings obtained with state transitions and the phosphorylation state of LHCII in Arabidopsis can be extrapolated to other species. Already between flowering plants and unicellular green algae like Chlamydomonas reinhardtii, fundamental regulatory mechanisms of photosynthesis are conserved. For instance, the LHCII kinase STN7 of Arabidopsis is conserved in C. reinhardtii, in which it is named Stt7. Because the diversification of the flowering plant species evolved relatively recently during evolution, it can be expected that the regulation of photosynthesis by LHCII (de)phosphorylation including the involved kinases and phosphatases exists in all flowering plant species.

“Chloroplast transit peptides” (cTP), as used herein, can be unambiguously determined by experimental proof by a person skilled in the art and can be predicted, based upon their sequence. The same applies to “transmembrane domains” as well as “protein phosphatase 2C signatures”. There exist various prediction algorithms for the subcellular localisation of a protein within a cell. The same is true for the prediction of potential transmembrane domains.

“siRNA”, as used herein, is meant to refer to small interfering RNA-molecules as known to someone skilled in the art. siRNA refers to, typically, double-stranded fragments of 15-21, 21-50, 50-100, 100-150, 150-200 ribonucleotides, with a few unpaired overhang bases on each end. These short double-stranded fragments are then separated into single strands and integrated into an active RISC complex. After integration into the RISC, siRNAs base pair to their target mRNA and induce cleavage of the mRNA, thereby preventing it from being used as a translation template. Usage of siRNAs is also envisaged in the context of the present invention to inhibit the activity of the aforementioned phosphatase of the light harvesting complex of photosystem II. The design of such siRNAs suitable for this purpose can be done by someone skilled in the art without undue experimentation.

The present invention also envisages mutants of the nucleic acid coding for the aforementioned phosphatase, which mutants are non-functional in that, either, the transcription of the nucleic acid coding sequence is switched off due to the presence of an additional regulatory sequence, or altering the coding sequence such that a non-functional transcript/protein is produced. These mutants can be introduced into a plant cell through standard techniques, such as transformation, shot gun approaches or agrobacterium tumefaciens mediated transformation. The transgenic plant cells thus obtained can then subsequently be regenerated into whole plants.

In the plant cells/plants in accordance with the present invention, the rate of photosynthesis is increased and/or the production of biomass is increased, both with reference to a plant cell/plant that has not been treated in accordance with the present invention.

Moreover, reference is made to the sequences in the enclosed sequence listing, wherein

SEQ ID NO:1 is the amino acid sequence of the phosphatase of the light harvesting complex of photosystem II in Arabidopsis thaliana,
SEQ ID NO:2 is the amino acid sequence of the phosphatase of the light harvesting complex of photosystem II in Vitis vinifera (common grape vine).
SEQ ID NO:3 is the amino acid sequence of the phosphatase of the light harvesting complex of photosystem II in Oryza sativa (rice).
SEQ ID NO:4 is the amino acid sequence of the phosphatase of the light harvesting complex of photosystem II in Hordeum vulgare (barley).
SEQ ID NO:5 is the amino acid sequence of the phosphatase of the light harvesting complex of photosystem II in Lycopersicon esculentum (tomato).
SEQ ID NO:6 is the amino acid sequence of the phosphatase of the light harvesting complex of photosystem II in Zea mays (corn).
SEQ ID NO:7 is the amino acid sequence of the phosphatase of the light harvesting complex of photosystem II in Triticum aestivum (wheat).
SEQ ID NO:8 is the amino acid sequence of the phosphatase of the light harvesting complex of photosystem II in Sorghum bicolor (Sorghum).
SEQ ID NO:9 is the amino acid sequence of the phosphatase of the light harvesting complex of photosystem II in Populus trichocarpa (cottonwood).
SEQ ID NO:10 is the amino acid sequence of the phosphatase of the light harvesting complex of photosystem II in Ricinus communis (castor oil plant).
SEQ ID NO:11 is the amino acid sequence of the phosphatase of the light harvesting complex of photosystem II in Picea sitchensis (Sitka spruce).
SEQ ID NO: 12-22 are the corresponding nucleic acid wildtype coding sequences of SEQ ID NO: 1-11.
SEQ ID NO: 23 is the amino acid sequence of the phosphatase of the light harvesting complex of photosystem II in Cucumis sativus (cucumber).
SEQ ID NO:24-47 are the amino acid sequences of the putative phosphatases (as obtained from an EST database) of Chinese cabbage (Brassica rapa)(24), rapeseed (Brassica napus)(25), tobacco (Nicotiana tabacum)(26), radish (Raphanus sativus)(27), orange (citrus sinensis)(28), cichory (Cichorium intybus)(29), wild radish (Raphanus raphanistrum)(30), potato (Solanum tuberosum)(31), Columbine species (Aquilegia sp.)(32), clementine (Citrus clementina)(33), common bean (Phaseolus vulgaris)(34), cow pea (Vigna unguiculata)(35), owl's clover species (Triphysaria sp., such as Triphysaria pusilla)(36), safflower (Carthamus tinctorius(37)), mandarin (Citrus reticulata)(38), lettuce (Lactuca sativa)(39), upland cotton (Gossypium hirsutum)(40), sunflower (Helianthus annuus)(41), soybean (Glycine max)(42), oat species such as Avena sativa or Avena barbata(43), switchgrass (Panicum virgatum)(44), annual wormwood (Artemisia annua)(45), onion (Allium cepa)(46), and nut species such as peanut (Arachis hypogaea)(47).
SEQ ID NO:48-72 are the corresponding nucleic acid wildtype coding sequences of SEQ ID NO: 23-47.

Moreover, reference is made to the figures, wherein

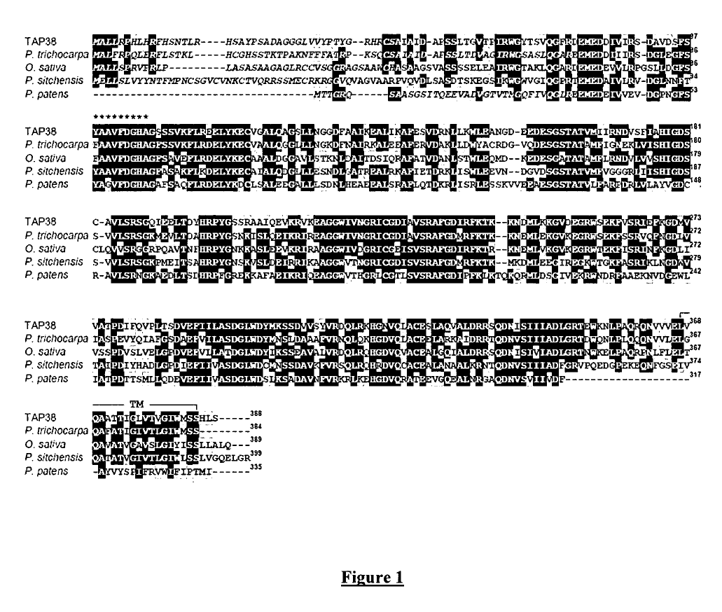

FIG. 1 shows a comparison of the TAP38 sequence with those of related proteins (homologues) from higher plants and moss,

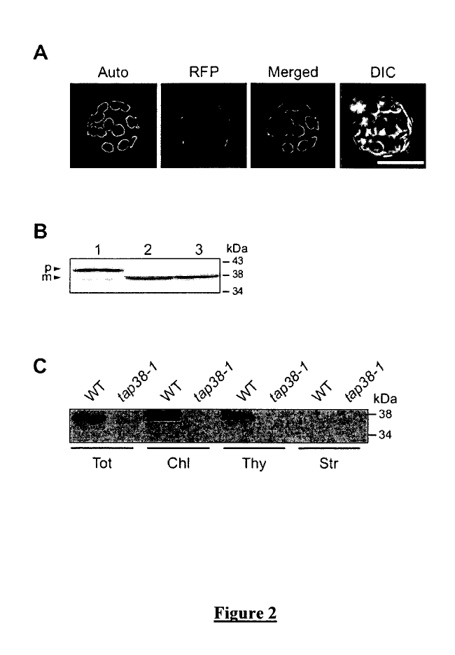

FIG. 2 shows the subcellular localization of TAP38;

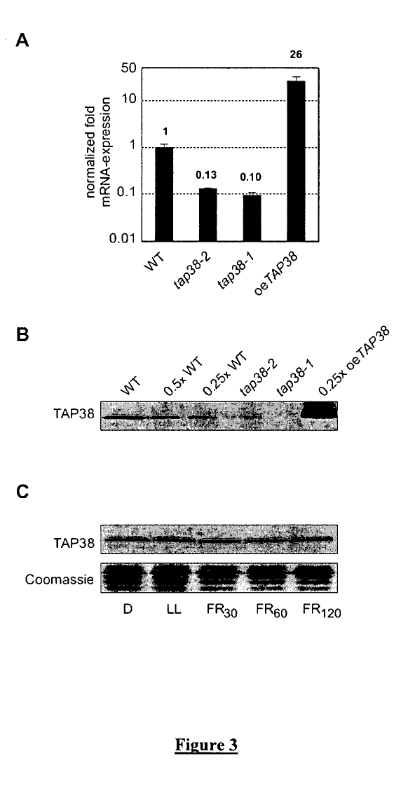

FIG. 3 shows the expression of TAP38 in tap38mutant, TAP38 overexpressor (oe TAP38) and wildtype (WT) plants;

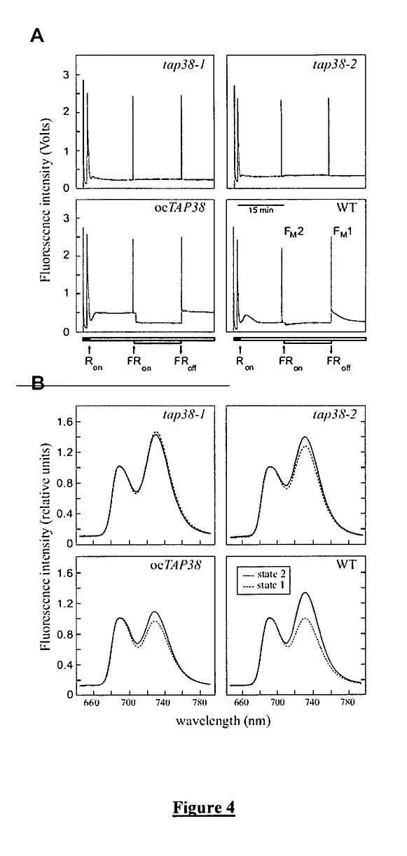

FIG. 4 shows that TAP38 is required for state transition;

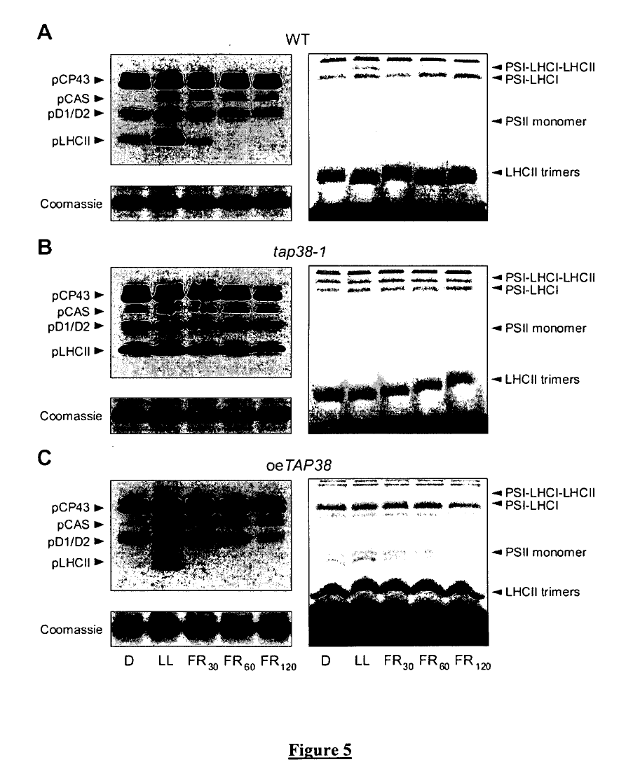

FIG. 5 shows that levels of phosphorylation of the light harvesting complex of photosystem II correlate inversely with TAP38 concentrations;

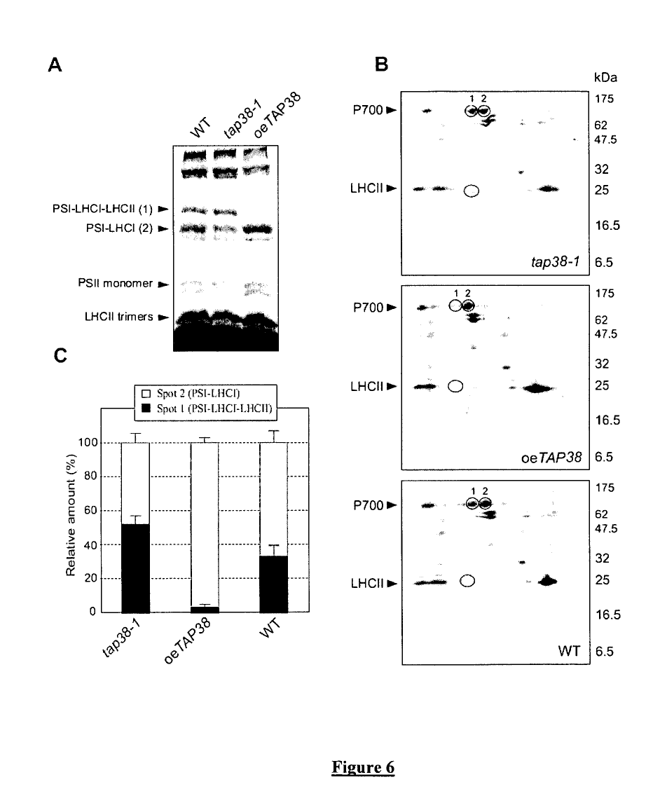

FIG. 6 shows a quantification of PSI-LHCI and PSI-LHCI-LHCII complexes under state 2 conditions; and

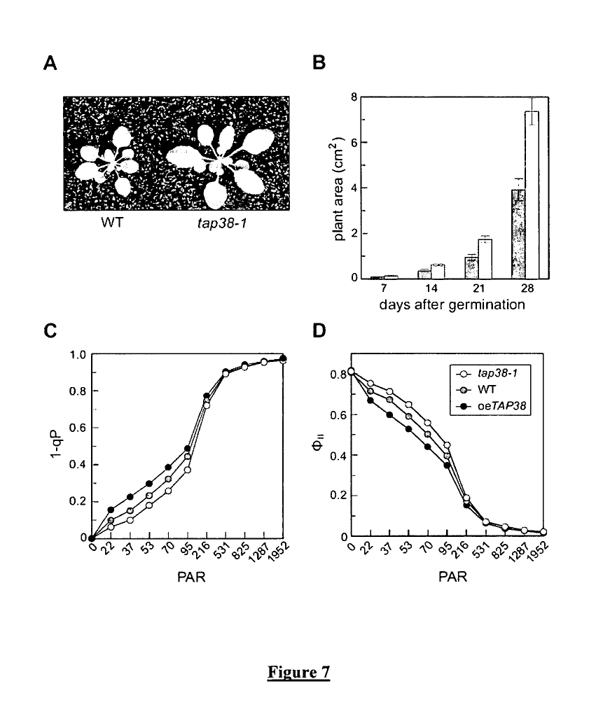

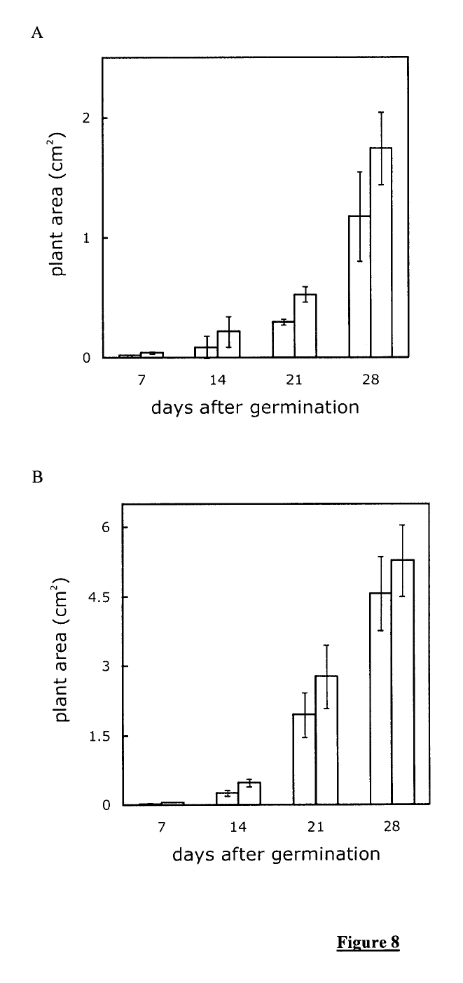

FIGS. 7 and 8 show the growth characteristics and photosynthetic performance of tap38 mutant plants under different conditions.

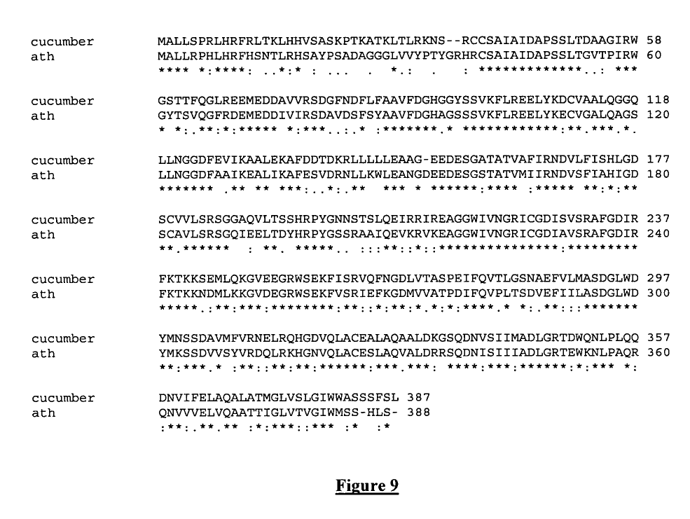

FIG. 9 shows a sequence alignment of the TAP38 sequence (“ath”) with the homologue sequence from cucumber (“cucumber”).

More specifically, the figures show the following:

FIG. 1. Comparison of the TAP38 sequence with those of related proteins from higher plants and moss.

The amino acid sequence of the Arabidopsis TAP38 protein (At4g27800) was compared with related sequences from Populus trichocarpa (POPTRDRAFT_{—}250893), Oryza sativa (Os01g0552300), Picea sitchensis (GenBank: EF676359.1) and Physcomitrella patens (PHYPADRAFT_{—}113608). Black boxes highlight strictly conserved amino acids, and gray boxes closely related ones. Amino acids that constitute the protein phosphatase 2C signature are indicated by asterisks. Putative chloroplast transit peptides (cTPs) are indicated in italics, and the potential transmembrane domain (TM) is highlighted.

FIG. 2. Subcellular localization of TAP38.

(A) Full-length TAP38-RFP was transiently expressed in Arabidopsis protoplasts and visualized by fluorescence microscopy. Auto, chlorophyll autofluorescence; RFP, fusion protein; merged, overlay of the two signals; DIC, differential interference contrast image. Scale bar, 50 μm.
(B) ³⁵S-labelled TAP38 protein, translated in vitro (lane 1, 10% translation product), was incubated with isolated chloroplasts (lane 2), which were subsequently treated with thermolysin to remove adhering precursor proteins (lane 3), prior to SDS-PAGE and autoradiography (p, precursor; m, mature protein).
(C) Immunoblot analyses of proteins from WT and tap38-1 leaves. Equal protein amounts were loaded. Tot, total protein; Chl, total chloroplasts; Thy, thylakoid proteins; Str, stromal proteins. Filters were immunolabelled with a TAP38-specific antibody.

FIG. 3. Expression of TAP38 in tap38 mutant, TAP38 overexpressor and WT plants.

(A) Quantification of TAP38 mRNAs by real-time PCR in WT, tap38-1, tap38-2 and oeTAP38 leaves using the primer combination 1-2 (as in Figures S1A and S1B).
(B) Thylakoid proteins from WT and tap38 mutants were loaded in the corresponding lanes. Reduced amounts of oeTAP38 thylakoids, corresponding to 25% of WT amount were loaded in the lane marked as 0.25×oeTAP38. Additionally, decreasing levels of WT thylakoids were loaded in the lanes indicated as 0.5×WT and 0.25×WT. Filters were immunolabelled with a TAP38-specific antibody raised against the mature TAP38 protein.
(C) Thylakoid membranes of WT plants exposed to different light conditions (see FIG. 5) were separated by SDS-PAGE. Immunodecoration of the corresponding western was performed using a TAP38 specific antibody raised against the mature protein. A detail of a replicate gel, corresponding to the LHCII migration region, stained with Coomassie is shown as loading control.

FIG. 4. TAP38 is required for state transitions.

(A) Red light (R) and red light supplemented with far-red (FR) light were used to induce transitions to state 2 and state 1, respectively. Fₘ1 and F_{M}2 represent maximal chlorophyll fluorescence levels in states 1 and 2, respectively. Horizontal bars indicate the length of illumination. Arrows point to the moment when the specific light is switched on/off. Traces are the average of 10 replicates.
(B) Low-temperature (77K) fluorescence emission spectra of thylakoids were recorded after exposure of plants to light inducing either state 1 (dashed lines) or state 2 (solid lines) (see also Experimental Procedures). The excitation wavelength was 475 nm and spectra were normalized with reference to peak height at 685 nm. Traces are the average of 10 replicates.

FIG. 5. Levels of LHCII phosphorylation correlate inversely with TAP38 concentrations. Left panel, Thylakoid proteins extracted from WT (A), tap38-1 (B) and oeTAP38 (C) plants kept in the dark (D, state 1), subsequently exposed to low light (LL, state 2), and then to far-red light for 30, 60 and 120 min (FR₃₀, FR₆₀, FR₁₂₀; state 1) were fractionated by SDS-PAGE. Phosphorylation of LHCII and PSII core proteins was detected by immunoblot analysis with a phosphothreonine-specific antibody. One out of three immunoblots for each genotype is shown

Right panel, Thylakoid proteins of WT (A), tap38-1 (B) and oeTAP38 (C) plants treated as in the left panel were subjected to BN-PAGE analysis. Accumulation of the state 2 associated 670-kDa protein complex correlates with the phosphorylation level of LHCII. Note that tap38-2 behaved very similarly to tap38-1 (data not shown). One out of three BN-PAGEs for each genotype is shown

FIG. 6. Quantification of PSI-LHCI and PSI-LHCI-LHCII complexes under state 2 conditions.

(A) BN PAGE of identical amounts of thylakoid proteins from WT, tap38-1 and oeTAP38 plants adapted to state 2 (low light; 80 μmol m⁻² s⁻¹). Bands representing the PSI-LHCI-LHCII (1) and PSI-LHCI (2) complexes are indicated.
(B) The WT, tap38-1 and oeTAP38 lanes from the BN-PAGE in (A) were fractionated further by denaturing 2D PAGE. Gels were stained with Coomassie. P700, photosystem I reaction centre; LHCII, light-harvesting complex of PSII (the bands indicative for the PSI-LHCI-LHCII (1) and PSI-LHCI (2) complexes are encircled).
(C) Densitometric quantification of the spots representing PSI-LHCI-LHCII (spot 1) and PSI-LHCI (spot 2) in panel B. Values are averages of three independent 2D gels for each genotype. Bars indicate standard deviations. Note that tap38-2 behaves very similarly to tap38-1 (data not shown).

FIG. 7. Growth characteristics and photosynthetic performance of climate chamber grown tap38 mutant plants.

(A) Phenotypes of 4-week-old tap38-1 and WT plants grown under low light conditions (80 μmol m⁻²s⁻¹) at an intermediate day length (12 h/12 h light/dark regime).
(B) Growth curve. Leaf areas of twenty plants of each genotype (WT, grey bars; tap83-1, white bars) were measured over a period of four weeks after germination. Mean values±standard deviations (SDs; bars) are shown.
(C-D), Measurements of light dependence of the photosynthetic parameters 1-qP (C) and effective quantum yield of PSII (VI; D) of plants grown as in panel A. WT, filled grey circles; tap38-1, open circles; oeTAP38, filled black circles; PAR, photosynthetically active radiation in μmol m⁻² s⁻¹. Average values were determined from five independent measurements (SD <5%). Note that tap38-2 and tap38-1 plants behave similarly. However, the tap38-2 mutant still synthesizes some TAP38 (see FIG. 3B), and its phenotype is slightly less severe.

FIG. 8. Growth characteristics of tap 38 mutant plants under short day (climate chamber) and greenhouse conditions.
(A) Growth curve of tap 38-1 and WT-plants grown under climate chamber conditions (100 μmol m⁻²s⁻¹) on a short day 8 h/16 h light/dark regime.
(B) Growth curve of tap 38-1 and WT-plants grown under greenhouse conditions on a 14 h/10 h light/dark regime. The leaf areas were measured over a period of 4 weeks after germination. WT (grey bars)=Wildtype; tap 38-1 (white bars)=tap 38-1-mutant.

FIG. 9. Sequence alignment of TAP38 sequence (ath) with the related homologue sequence from cucumber (“cucumber”).

The invention will now be further described by the following examples, which are given to illustrate the invention, not to limit it.

### EXAMPLES

### Example 1

### Plant Material and Growth Measurements

Procedures for plant propagation and growth measurements have been described elsewhere [44]. The tap38-2 insertion line (SALK_{—}025713) was identified in the SALK collection [34] (http://signal.salk.edu/), whereas insertion line tap38-1 (SAIL_{—}514_C03) originated from the Sail collection [33]. Both lines were identified by searching the insertion flanking database SIGNAL (http://signal.salk.edu/cgi-bin/tdna express). To generate oeTAP38 lines the coding sequence of TAP38 was cloned into the plant expression vector pH2GW7 (Invitrogen). For complementation of the tap38-1 mutant the TAP38 genomic DNA, together with 1 kb of its natural promoter was ligated into the plant expression vector pP001-VS. The constructs were used to transform flowers of Col-0 or tap38-1 mutant plants by the floral dipping technique as described [45]. Transgenic plants, after selection for resistance to hygromycin (oeTAP38) or Basta herbicide (complemented tap38-1), were grown on soil in a climate chamber under controlled conditions (PAR: 80 μmol m⁻² s⁻¹, 12/12 h dark/light cycles). The T2 generation of the oeTAP38 plants was used for the experiments reported. Successful complementation of tap38-1 mutants was confirmed by measurements of chlorophyll fluorescence and LHCII phosphorylation levels under light regimes promoting state transitions. For growth measurements, WT and tap38-1 mutant plants were grown either under controlled climate chamber (PAR: 80 or 100 μmol m⁻² s⁻¹, 12/12 h or 8/16 h dark/light cycles) or greenhouse conditions (14/10 h dark/light regime).

Subcellular Localization of the TAP38-dsRED Fusion in Arabidopsis Protoplasts

The full-length coding region of the TAP38 gene was cloned into the vector pGJ1425, in frame with, and immediately upstream of the sequence encoding dsRED [32]. Isolation, transfection and fluorescence microscopy of A. thaliana protoplasts were performed as described [46].

In-vitro import of TAP38 into pea chloroplasts The coding region of TAP38 was cloned into the pGEM-Teasy vector (Promega) downstream of its SP6 promoter region, and mRNA was produced in vitro using SP6 RNA polymerase (MBI Fermentas). The TAP38 precursor protein was synthesized in a Reticulocyte Extract System (Flexi®, Promega) in the presence of [³⁵S]methionine. Aliquots of the translation reaction were incubated with intact chloroplasts, and protein uptake was analysed after treatment of isolated chloroplasts with thermolysin (Calbiochem) as described previously [47]. Labelled proteins were subjected to SDS-PAGE and detected by phosphorimaging (Typhoon; Amersham Biosciences).

cDNA Synthesis, Semi-Quantitative Reverse-Transcriptase PCR and Real-Time PCR

Total RNA was extracted with the RNeasy Plant Mini Kit (QIAGEN) according to the manufacturer's instructions. cDNA was prepared from 1 μg of total RNA using the iScript cDNA Synthesis Kit (Bio-Rad) according to the manufacturer's instructions. For semi-quantitative reverse-transcriptase PCR, cDNA was diluted 10-fold, and 3 μl of the dilution was used in a 20 μl reaction. Thermal cycling consisted of an initial step at 95° C. for 3 mM, followed by 30 cycles of 10 s at 95° C., 30 s at 55° C. and 10 s at 72° C. For real-time PCR analysis, 3 μl of the diluted cDNA was mixed with iQ SYBR Green Supermix (Bio-Rad). Thermal cycling consisted of an initial step at 95° C. for 3 min, followed by 40 cycles of 10 s at 95° C., 30 s at 55° C. and 10 s at 72° C., after which a melting curve was performed. Real-time PCR was monitored using the iQ5™ Multi Color Real-Time PCR Detection System (Bio-Rad). All reactions were performed in triplicate with at least two biological replicates.

### Protein Isolation and Immunoblot Analysis

Total protein extracts and proteins from total chloroplasts, thylakoids and the stroma fraction were prepared from 4-week-old leaves in the presence of 10 mM NaF as described [46,48]. Immunoblot analyses with phosphothreonine-specific antibodies (Cell Signaling) or polyclonal antibodies raised against the mature TAP38 protein were performed as described [43].

### Blue-Native (BN) PAGE and 2D Polyacrylamide Gel Electrophoresis (2D PAGE)

For BN PAGE, thylakoid membranes were prepared as described above. Aliquots corresponding to 100 pg of chlorophyll were solubilised in solubilisation buffer (750 mM 6-aminocaproic acid; 5 mM EDTA, pH 7; 50 mM NaCl; 1.5% digitonin) for 1 h at 4° C. After centrifugation for 1 h at 21,000 g, the solubilised material was fractionated by non-denaturing BN-PAGE at 4° C. as described [37].

For 2D PAGE, samples were fractionated in the first dimension by BN-PAGE as described above and subsequently by denaturing sodium dodecyl sulfate PAGE as described previously [49]. Densitometric analysis of the stained gels was performed using the Lumi Analyst 3.0 (Boehringer, Mannheim, Germany).

Measurements of state transitions and 77K fluorescence State transitions were measured by pulse amplitude modulation fluorometry [35] and 77K fluorescence emission analysis [12,36]. For state transition measurements, five plants of each genotype were analysed, and mean values and standard deviations were calculated. In-vivo chlorophyll a fluorescence of single leaves was measured using the Dual-PAM 100 (Walz). Pulses (0.5 s) of red light (5000 μmol m⁻² s⁻¹) were used to determine the maximum fluorescence and the ratio (F_{M}−F₀)/F_{M}=F_{V}/F_{M}. Quenching of chlorophyll fluorescence due to state transitions (qT) was determined by illuminating dark-adapted leaves with red light (35 μmol m⁻² s⁻¹, 15 min) and then measuring the maximum fluorescence in state 2 (F_{M}2). Next, state 1 was induced by adding far-red light (maximal light intensity corresponding to level 20 in the Dual-PAM setting, 15 min) and recording F_{M}1. qT was calculated as: qT=(F_{M}1−F_{M}2)/F_{M}2 [35].

For 77K fluorescence emission spectroscopy the fluorescence spectra of thylakoids were recorded after irradiating plants with light that favoured excitation of PSII (80 μmol m⁻² s⁻¹, 8 h) or PSI (LED light of 740 nm wavelength, 4.6 μmol m⁻² s⁻¹, 2 h). Thylakoids were isolated in the presence of 10 mM NaF as described [11], and 77 K fluorescence spectra were obtained by excitation at 475 nm using a Spex Fluorolog mod.1 fluorometer (Spex Industries, Texas, USA). The emission between 600 and 800 nm was recorded, and spectra were normalized relative to peak height at 685 nm.

### Example 2

### Screening for Candidate LHCII Phosphatases

To identify the LHCII phosphatase, the present inventors systematically isolated loss-of-function mutants for known chloroplast protein phosphatases and assessed their capacity to dephosphorylate LHCII (see below for details). However, none of the nine protein phosphatases At3g52180 (DSP4/SEX4), At4g21210 (AtRP1), At1g07160, At3g30020, At4g33500, At1g67820, At2g30170, At3g10940, At4g03415, demonstrated to reside in the chloroplast [25-29], qualified as the LHCII phosphatase (data not shown). Next they extended their search to protein phosphatases tentatively identified as chloroplast proteins by proteomic analyses in A. thaliana [30,31]. Of those, At4g27800 turned out to be the most promising candidate.

At4g27800 (TAP38) is a Thylakoid-Associated Protein Phosphatase

Proteins with high homology to At4g27800 exist in mosses and higher plants, but not in algae or prokaryotes. Furthermore, At4g27800 and its homologues share a predicted N-terminal chloroplast transit peptide (cTP), a putative transmembrane domain (TM) at their very C-terminus and a protein phosphatase 2C signature (FIG. 1).

In protoplasts transfected with At4g27800 fused to the coding sequence for the Red Fluorescent Protein (RFP) [32], the fusion protein localized to chloroplasts (FIG. 2A). Chloroplast import assays with the radioactively labelled At4g27800 protein confirmed the uptake into the chloroplast with concomitant removal of its cTP. Mature At4g27800 has a molecular weight of ˜38 kDa (FIG. 2B). Immunoblot analysis using a specific antibody raised against the mature At4g27800 protein (FIG. 2C) detected the protein in thylakoid preparations but not in stromal fractions. At4g27800 was renamed TAP38 ('Thylakoid-Associated Phosphatase of 38 kDa').

TAP38 Expression in Wild Type, tap38 Mutant and TAP38 Overexpressor Plants

Two tap38 insertion mutants, tap38-1 (SAIL_{—}14_C03) [33] and tap38-2 (SALK_{—}025713) [34], were obtained from T-DNA insertion collections (Figure S1A). In tap38-1 and tap38-2 plants, amounts of TAP38 transcripts were severely reduced—to 10% and 13% of WT levels, respectively (FIG. 3A). Conversely, in transgenic lines carrying the TAP38 coding sequence under control of the ³⁵S promoter of Cauliflower Mosaic Virus (oeTAP38), levels of TAP38 mRNA were much higher than in wild type (WT) (FIG. 3A). TAP38 protein concentrations reflected the abundance of TAP38 transcripts: tap38-1 and tap38-2 thylakoids had <5% and ˜10% of WT levels, respectively, while the oeTAP38 plants displayed >20-fold overexpression on the protein level (FIG. 3B). TAP38 protein levels were also determined under light conditions relevant for state transitions (see Example 1). In WT plants, TAP38 was constitutively expressed at similar levels under all light conditions applied (FIG. 3C).

### TAP38 is Required for State Transitions

To determine whether TAP38 is involved in state transitions, chlorophyll fluorescence was measured in WT, tap38 and oeTAP38 leaves (FIG. 4A). Plants were exposed to light conditions that stimulate either state 2 (red light) or state 1 (far-red light) [35], and the corresponding maximum fluorescence F_{M}2 (state 2) and Fₘ1 (state 1) values were determined. Because the light intensity chosen to induce state transitions did not elicit photoinhibition (as monitored by measurements of F_{V}/Fₘ—the maximum quantum yield; data not shown), changes in F_{M}, the maximum fluorescence, could be attributed to state transitions alone. This allowed us to calculate qT [35], the degree of quenching of chlorophyll fluorescence due to state transitions. In the tap38 mutants, qT was markedly decreased (tap38-1, 0.01±0.004; tap38-2, 0.02±0.004; WT, 0.11±0.001). In tap38-1 plants complemented with the TAP38 genomic sequence (including its native promoter), qT values were normal (data not shown), confirming that state transitions require TAP38. Interestingly, oeTAP38 plants exhibited qT values of about 0.01±0.001, indicating that both absence and excess of TAP38 interfere with the ability to undergo normal state transitions.

To determine the antenna sizes of PSII and PSI, 77K fluorescence emission spectra were measured under state 1 (exposure to far-red light) and state 2 (low light) conditions as described [11,12,36] (FIG. 4B). The spectra were normalized at 685 nm, the peak of PSII fluorescence. In WT, the transition from state 1 to state 2 was accompanied by a marked increase in relative PSI fluorescence at 730 nm, reflecting the redistribution of excitation energy from PSII to PSI. In contrast, in tap38 leaves, the PSI fluorescence peak was relatively high even under state 1-promoting conditions, implying that the mutants were blocked in state 2—i.e. pLHCII should be predominantly attached to PSI. Additionally, under state 2-promoting light conditions, the PSI antenna size (expressed as F₇₃₀/F₆₈₅) was larger in tap38 mutants than in WT (tap38.1, 1.47; tap38.2, 1.45; WT, 1.38; see also Table S1), arguing in favour of the idea that in tap38 plants a larger fraction of the mobile pool of LHCII can attach to PSI. On the contrary, in oeTAP38 plants the relative fluorescence of PSI hardly increased at all under conditions expected to induce the state 1→state 2 shift (FIG. 4B; Table S1). This behaviour resembles that of stn 7 mutants, which are blocked in state 1, i.e. with LHCII permanently attached to PSII [12].

Levels of LHCII phosphorylation correlate inversely with TAP38 concentrations

It is generally accepted that state transitions require reversible phosphorylation of LHCII [2,4,5]. Therefore, the phosphorylation state of LHCII was monitored under light conditions that favour state 1 (dark or far-red light treatment) or state 2 (low light). Plants with abnormal levels of TAP38 and WT plants were dark adapted for 16 h (state 1), then exposed to low light (80 μmol m⁻²s⁻¹, 8 h) (state 2), and then to far-red light (4.5 μmol m⁻² s⁻¹, 740 nm) for up to 120 min to induce a return to state 1. Thylakoid proteins were isolated after each treatment, fractionated by SDS-PAGE, and analysed with a phosphothreonine-specific antibody (FIG. 5, left panels). WT plants showed the expected increase in phosphorylated LHCII (pLHCII) during the transition from state 1 (dark, D) to state 2 (low light, LL), followed by a progressive decrease in pLHCII upon exposure to far-red light (FR). In tap38 mutants, levels of pLHCII were aberrantly high at all time points, while the oeTAP38 plants again mimicked the stn? phenotype [9,12], displaying constitutively reduced levels of pLHCII.

### Quantification of PSI-LHCI-LHCII Complex Formation Under Varying TAP38 Concentrations

To directly visualize how alterations in LHCII phosphorylation in lines lacking or overexpressing TAP38 affect the distribution of the mobile LHCII fraction between the two photosystems, we subjected thylakoid protein complexes of plants adapted to state 1 (dark and far-red light treatments) or state 2 (low light treatment) to non-denaturing Blue-native (BN) PAGE [37] (FIG. 5, right panels). In this assay, a pigment-protein complex of about 670 kDa, which represents pLHCII associated with the PSI-LHCI complex, can be visualized [14,37,38]. Whereas in WT thylakoids the 670-kDa complex was only observable under state 2 conditions (FIG. 5A, right panel), as previously reported [14,38], the constitutive phosphorylation of LHCII in the tap38 mutants was associated with the presence of a prominent band for the 670-kDa complex under all light conditions (FIG. 5B, right panel). The formation of the 670-kDa complex was totally prevented in oeTAP38 plants with a block in state 1 and highly reduced levels of pLHCII (FIG. 5C, right panel). Two-dimensional (2D) PA gel fractionation confirmed that the pigment-protein complex consists of PSI and LHCI subunits, together with a portion of pLHCII that associates with PSI upon state 1→state 2 transition in WT plants (FIG. 6B). Additionally, quantification of the different PSI complexes on 2D PA gels showed that the number of PSI complexes associated with LHCII was increased in the tap38 mutants (FIG. 6C), supporting the findings obtained from the 77K fluorescence analyses.

Plants without TAP38 Show Improved Photosynthesis and Growth

When kept at an intermediate daylength (12/12h light/dark cycles) in climate chambers under low light intensities (80 μmol m⁻² s⁻¹) that favour state 2, tap38 mutants grew larger than WT plants (FIG. 7A), whereas oeTAP38 plants behaved like WT (data not shown). Detailed growth measurements revealed that the tap38 mutants exhibited a constant growth advantage over WT plants, starting at the cotyledon stage (FIG. 7B). Similar results in terms of a growth advantage over WT plants were also obtained for short day conditions (100 μmol m⁻² s⁻¹ at an 8/16h light/dark regime) in climate chambers, as well as under greenhouse conditions (14/10h light/dark cycles) (see FIGS. 8A and 8B). Because this difference might be attributable to altered photosynthetic performance, parameters of thylakoid electron flow were measured. The fraction of Q_{A} (the primary electron acceptor of PSII) present in the reduced state (1-qP) was lower in tap38-1 plants (0.06±0.01) than in WT (0.10±0.01), when both genotypes were grown as in FIG. 7A and chlorophyll fluorescence was excited with 22 μmol m⁻² s⁻¹ actinic red light. Comparable differences in the redox state of the primary electron acceptor persisted up to 95 μmol m⁻² s⁻¹ actinic red light (FIG. 7C), indicating that the tap38-1 mutant can redistribute a larger fraction of energy to PSI, in accordance with the increase in its antenna size under state 2 (see FIG. 4B; Table S1 and FIG. 6). This idea was supported by measurements of the maximum (F_{V}/F_{M}) and effective (Φ_{II}) quantum yields of PSII. F_{V}/F_{M} remained unaltered in mutant plants (see FIG. 7D, dark-adapted plants, PAR=0), indicating WT-like efficiency of mutant PSII complexes. However, Φ_{II} was increased in tap38-1 (0.75±0.01) relative to WT (0.72±0.01), suggesting that electron flow through the thylakoids was more efficient in tap38-1 (FIG. 7D). The improvement in photosynthetic performance of the tap38 mutants was most pronounced under low and moderate illumination (FIGS. 7C and 7D), as expected from their growth phenotype.

### Possible Modes of Action of Tap38

How does TAP38 control LHCII dephosphorylation? Three possibilities appear plausible: 1) TAP38 negatively regulates the activity of STN7 (e.g. by dephosphorylating it), 2) TAP38 dephosphorylates LHCII directly, or 3) forms part of a phosphorylation/dephosphorylation cascade that controls the activity of the LHCII kinase or phosphatase. The observation that oeTAP38 plants, although showing a >20-fold increase in TAP38 levels, still exhibit residual LHCII phosphorylation (see FIG. 5C), together with the fact that reversible phosphorylation of STN7 has not been demonstrated [39], strongly argues against the idea that TAP38 does inhibit STN7 by dephosphorylation. Differences in TAP38 levels resulted in a clear change in pLHCII levels: while in tap38 mutants a strong reduction in TAP38 led to a constantly high level of pLHCII, strong overexpression of TAP38 (oeTAP38) severely reduced the amount of pLHCII. However, in WT where pLHCII levels can vary dramatically depending on the light conditions [9,12] (see also FIG. 5A), TAP38 seems to be constitutively expressed under the different light conditions applied (see FIG. 3C). A plausible explanation for this is that TAP38 is constitutively active and directly responsible for the dephosphorylation of LHCII. For that, TAP38 would need to be present in a certain concentration range (as it is the case for WT) to constantly dephosphorylate LHCII. In agreement with that, thylakoid protein phosphatase reactions have been described as redox-independent, leading to the conclusion that the redox dependency of LHCII phosphorylation is a property of the kinase reaction [40]. This, together with the observation that Stt7 levels increase under prolonged state 2 conditions (favouring LHCII phosphorylation) and decrease under state 1 conditions (favouring dephosphorylation of LHCII) [17], argues in favour of the hypothesis that the LHCII kinase is the decisive factor in controlling the phosphorylation state of LHCII. Despite the obvious TAP38 dosage dependence of LHCII dephosphorylation (see FIGS. 5 and 6), TAP38 activity could be regulated on other levels than only its abundance. However, the strong decrease or increase of TAP38 levels in tap38 mutant and oeTAP38 plants might interfere with other types of regulation in these genotypes.

### Is TAP38 the Long-Sought LHCII Phosphatase?

As outlined above, the dependence of LHCII dephosphorylation upon TAP38 dosage—when comparing tap38 mutants, WT and TAP38 overexpressors—strongly suggests that TAP38 dephosphorylates LHCII directly. Alternatively, TAP38 could act in a phosphorylation/dephosphorylation cascade that controls the activity of the LHCII phosphatase. Although the latter hypothesis cannot be excluded, a set of evidences point to a direct role of TAP38 on LHCII phosphorylation. Indeed, as in the case of STN kinases, extensive efforts searching to identify other LHCII phosphatase candidates failed: knout-out lines for all the protein phosphatases demonstrated to be located in the chloroplast [25-29] did not show any alteration in LHCII phosphorylation. Additionally, extensive biochemical studies did not reveal the existence of a complex network of phosphatases involved in LHCII dephosphorylation, but postulated the involvement of only two distinct chloroplast protein phosphatases from different families in the dephosphorylation of thylakoid phosphoproteins [20-23,41]. Our data support this notion, as shown by the absence of major alterations in the phosphorylation pattern of CP43, D1 and D2 subunits in tap38 mutant plants (see FIG. 5). Moreover, LHCII dephosphorylation was suggested to be catalysed by only two independent protein phosphatases, a membrane bound-one and a stromal protein phosphatase [41]. In contrast to this, our results clearly show that TAP38, a thylakoid-associated phosphatase, alone is responsible for LHCII dephosphorylation. Thus, although slightly leaky, the tap38-1 mutants show a large fraction of LHCII in the phosphorylated state under all investigated conditions (see FIG. 5). If a second LHCII phosphatase with redundant function would operate in chloroplasts, one would expect some residual dephosphorylation of LHCII. A plausible explanation for the previously shown stromal LHCII dephosphorylation activity [20] might be that during the preparation of stromal extracts a significant portion of TAP38 was released from the thylakoid membrane into the stroma.

Taken these observations together, it appears likely that, like in the case of the STN kinases, two distinct phosphatases are needed to dephosphorylate LHCII and PSII core proteins. TAP38, similar to the STN7 kinase, seems to have a high specificity for LHCII as substrate. The counterpart of STN8 [9,42]—the PSII-core specific phosphatase, remains to be identified. However, as in the case of the STN7 and STN8 kinases, some degree of substrate overlap seems to exist also between the phosphatases, as shown by the more rapid dephosphorylation of D1/D2 subunits in the TAP38 overexpressor lines exposed to far-red light conditions (see FIG. 5C).

Uncoupling of LHCII Phosphorylation from PQ Redox State

It is known that an increase in the relative size of the reduced fraction of the plastoquinone pool (PQH₂) enhances phosphorylation of LHCII [1,5,38,43]. Depletion of TAP38 in tap38 mutants, however, increases both LHCII phosphorylation (see FIG. 5B) and PQ oxidation (see 1-qP values in FIG. 7C). This discrepancy can be resolved by assuming that the enhanced oxidation of PQ caused by the increase in PSI antenna size (and LHCII phosphorylation) in tap38 plants is not sufficient to down-regulate the LHCII kinase to such an extent that it can compensate for the decline in LHCII dephosphorylation.

### How can Absence of TAP38 Improve Photosynthesis and Growth?

The enhanced photosynthetic performance indicated by an increase in Φ_{II} and a decrease of 1-qP (see FIG. 7D), as well as the growth advantage of the tap38 mutants under constant moderate light intensities that stimulate LHCII phosphorylation and state 2, can be attributed to the redistribution of a larger fraction of energy to PSI. This is in accordance with the increase in PSI antenna size in tap38 mutants when compared to WT plants (see FIG. 4B, Table S1 and FIG. 6). Therefore, it is straightforward to speculate that the enhanced PSI antenna size provides the tap38 mutants with a more robust photosynthetic electron flow under conditions that preferentially excite PSII and induce state 2. As a consequence of the more balanced light reaction, the photosynthetic efficiency is improved resulting in an increased growth rate. However, the fitness advantage will revert under conditions that induce state 1, or under more natural conditions with fluctuating light; here, it can be expected that tap38 mutants perform less efficient than the WT with respect to photosynthesis and growth, very similar to what has been observed for the stn? mutant [12,13].

TABLE S1
Energy distribution between PSI and PSII measured as the fluorescence
emission ratio at 730 nm and 685 nm (F₇₃₀/F₆₈₅)

F₇₃₀/F₆₈₅

| | State 1 | State 2 |
|---|---|---|
| WT | 0.99 ± 0.05 | 1.38 ± 0.03 |
| tap38-1 | 1.49 ± 0.04 | 1.47 ± 0.02 |
| tap38-2 | 1.32 ± 0.03 | 1.45 ± 0.04 |
| oeTAP38 | 0.98 ± 0.02 | 1.11 ± 0.03 |

The features of the present invention disclosed in the specification, the claims, and/or in the accompanying drawings may, both separately and in any combination thereof, be material for realizing the invention in various forms thereof.

### LIST OF REFERENCES

1. Allen J F, Forsberg J (2001) Molecular recognition in thylakoid structure and function. Trends Plant Sci 6: 317-326.
2. Eberhard S, Finazzi G, Wollman F A (2008) The dynamics of photosynthesis. Annu Rev Genet. 42: 463-515.
3. Haldrup A, Jensen P E, Lunde C, Scheller H V (2001) Balance of power: a view of the mechanism of photosynthetic state transitions. Trends Plant Sci 6: 301-305.
4. Rochaix J D (2007) Role of thylakoid protein kinases in photosynthetic acclimation. FEBS Lett 581: 2768-2775.
5. Wollman F A (2001) State transitions reveal the dynamics and flexibility of the photosynthetic apparatus. EMBO J. 20: 3623-3630.
6. Allen J F, Race H L (2002) Will the real LHC II kinase please step forward? Sci STKE 2002: PE43.
7. Allen J F (1992) Protein phosphorylation in regulation of photosynthesis. Biochim Biophys Acta 1098: 275-335.
8. Delosme R, Beal D, Joliot P (1994) Photoacoustic detection of flash-induced charge separation in photosynthetic systems—spectral dependence of the quantum yield. Biochim Biophys Acta 1185: 56-64.
9. Bonardi V, Pesaresi P, Becker T, Schleiff E, Wagner R, et al. (2005) Photosystem II core phosphorylation and photosynthetic acclimation require two different protein kinases. Nature 437: 1179-1182.
10. Lunde C, Jensen P E, Haldrup A, Knoetzel J, Scheller H V (2000) The PSI-H subunit of photosystem I is essential for state transitions in plant photosynthesis. Nature 408: 613-615.
11. Tikkanen M, Piippo M, Suorsa M, Sirpio S, Mulo P, et al. (2006) State transitions revisited-a buffering system for dynamic low light acclimation of Arabidopsis. Plant Mol Biol 62: 779-793.
12. Bellafiore S, Barneche F, Peltier G, Rochaix J D (2005) State transitions and light adaptation require chloroplast thylakoid protein kinase STN7. Nature 433: 892-895.
13. Frenkel M, Bellafiore S, Rochaix J D, Jansson S (2007) Hierarchy amongst photosynthetic acclimation responses for plant fitness. Physiol Plant 129: 455-459.
14. Pesaresi P, Hertle A, Pribil M, Kleine T, Wagner R, Strissel H, Ihnatowicz A, Bonardi V, Scharfenberg M, Schneider A, et al. (2009) Arabidopsis STN7 kinase provides a link between short- and long-term photosynthetic acclimation. Plant Cell (in press).
15. Vener A V, VanKan P J M, Rich P R, Ohad I, Andersson B (1997) Plastoquinol at the quinol oxidation site of reduced cytochrome b f mediates signal transduction between light and protein phosphorylation: Thylakoid protein kinase deactivation by a single-turnover flash. Proc Natl Acad Sci USA 94: 1585-1590.
16. Zito F, Finazzi G, Delosme R, Nitschke W, Picot D, et al. (1999) The Qo site of cytochrome b₆f complexes controls the activation of the LHCII kinase. EMBO J. 18: 2961-2969.
17. Lemeille S, Willig A, Depege-Fargeix N, Delessert C, Bassi R, et al. (2009) Analysis of the chloroplast protein kinase Stt7 during state transitions. PLoS Biol 7: e45.
18. Rintamaki E, Martinsuo P, Pursiheimo S, Aro E M (2000) Cooperative regulation of light-harvesting complex II phosphorylation via the plastoquinol and ferredoxin-thioredoxin system in chloroplasts. Proc Natl Acad Sci USA 97: 11644-11649.
19. Depege N, Bellafiore S, Rochaix J D (2003) Role of chloroplast protein kinase Stt7 in LHCII phosphorylation and state transition in Chlamydomonas. Science 299: 1572-1575.
20. Hammer M F, Sarath G, Markwell J (1995) Dephosphorylation of the thylakoid membrane light-harvesting complex-II by a stromal protein phosphatase. Photosynth Res 45: 195-201.
21. Hast T, Follmann H (1996) Identification of two thylakoid-associated phosphatases with protein phosphatase activity in chloroplasts of the soybean (Glycine max). J Photochem Photobiol B—Biol 36: 313-319.
22. Sun G, Bailey D, Jones M W, Markwell J (1989) Chloroplast thylakoid protein phosphatase is a membrane surface-associated activity. Plant Physiol 89: 238-243.
23. Vener A V, Rokka A, Fulgosi H, Andersson B, Herrmann R G (1999) A cyclophilin-regulated PP2A-like protein phosphatase in thylakoid membranes of plant chloroplasts. Biochemistry 38: 14955-14965.
24. Cohen P (1989) The structure and regulation of protein phosphatases. Annu Rev Biochem 58: 453-508.
25. Chastain C J, Xu W, Parsley K, Sarath G, Hibberd J M, et al. (2008) The pyruvate, orthophosphate dikinase regulatory proteins of Arabidopsis possess a novel, unprecedented Ser/Thr protein kinase primary structure. Plant J 53: 854-863.
26. Kerk D, Conley T R, Rodriguez F A, Tran H T, Nimick M, et al. (2006) A chloroplast-localized dual-specificity protein phosphatase in Arabidopsis contains a phylogenetically dispersed and ancient carbohydrate-binding domain, which binds the polysaccharide starch. Plant J 46: 400-413.
27. Niittyla T, Comparot-Moss S, Lue W L, Messerli G, Trevisan M, et al. (2006) Similar protein phosphatases control starch metabolism in plants and glycogen metabolism in mammals. J Biol Chem 281: 11815-11818.
28. Schliebner I, Pribil M, Zuhlke J, Dietzmann A, Leister D (2008) A survey of chloroplast protein kinases and phosphatases in Arabidopsis thaliana. Curr Genomics 9: 184-190.
29. Sokolov L N, Dominguez-Solis J R, Allary A L, Buchanan B B, Luan S (2006) A redox-regulated chloroplast protein phosphatase binds to starch diurnally and functions in its accumulation. Proc Natl Acad Sci USA 103: 9732-9737.
30. Sun Q, Zybailov B, Majeran W, Friso G, Olinares P D, et al. (2009) PPDB, the Plant Proteomics Database at Cornell. Nucleic Acids Res 37: D969-974.
31. Zybailov B, Rutschow H, Friso G, Rudella A, Emanuelsson 0, et al. (2008) Sorting signals, N-terminal modifications and abundance of the chloroplast proteome. PLoS ONE 3: e 1994.
32. Jach G, Binot E, Frings S, Luxa K, Schell J (2001) Use of red fluorescent protein from Discosoma sp. (dsRED) as a reporter for plant gene expression. Plant J 28: 483-491.
33. Sessions A, Burke E, Presting G, Aux G, McElver J, et al. (2002) A high-throughput Arabidopsis reverse genetics system. Plant Cell 14: 2985-2994.
34. Alonso J M, Stepanova A N, Leisse T J, Kim C J, Chen H, et al. (2003) Genome-wide insertional mutagenesis of Arabidopsis thaliana. Science 301: 653-657.
35. Jensen P E, Gilpin M, Knoetzel J, Scheller H V (2000) The PSI-K subunit of photosystem I is involved in the interaction between light-harvesting complex I and the photosystem I reaction center core. J Biol Chem 275: 24701-24708.
36. Tikkanen M, Nurmi M, Suorsa M, Danielsson R, Mamedov F, et al. (2008) Phosphorylation-dependent regulation of excitation energy distribution between the two photosystems in higher plants. Biochim Biophys Acta 1777: 425-432.
37. Heinemeyer J, Eubel H, Wehrnhoner D, Jansch L, Braun HP (2004) Proteomic approach to characterize the supramolecular organization of photosystems in higher plants. Phytochemistry 65: 1683-1692.
38. Pesaresi P; Lunde C, Jahns P, Tarantino D, Meurer J, et al. (2002) A stable LHCII-PSI aggregate and suppression of photosynthetic state transitions in the psael-1 mutant of Arabidopsis thaliana. Planta 215: 940-948.
39. Heazlewood J L, Durek P, Hummel J, Selbig J, Weckwerth W, et al. (2008) PhosPhAt: a database of phosphorylation sites in Arabidopsis thaliana and a plant-specific phosphorylation site predictor. Nucleic Acids Res 36: D1015-1021.
40. Silverstein T, Cheng L, Allen J F (1993) Chloroplast thylakoid protein phosphatase reactions are redox-independent and kinetically heterogeneous. FEBS Lett 334: 101-105.
41. Hammer M F, Sarath G, Osterman J C, Markwell J (1995) Assesing modulations of stromal and thylakoid light-harvesting complex-II phosphatase activites with phosphopeptide substrates. Photosynth Res 44: 107-115.
42. Vainonen J P, Hansson M, Vener A V (2005) STN8 protein kinase in Arabidopsis thaliana is specific in phosphorylation of photosystem II core proteins. J Biol Chem 280: 33679-33686.
43. Ihnatowicz A, Pesaresi P, Lohrig K, Wolters D, Muller B, et al. (2008) Impaired photosystem I oxidation induces STN7-dependent phosphorylation of the light-harvesting complex I protein Lhca4 in Arabidopsis thaliana. Planta 227: 717-722.
44. Leister D, Varotto C, Pesaresi P, Niwergall A, Salamini F (1999) Large-scale evaluation of plant growth in Arabidopsis thaliana by non-invasive image analysis. Plant Physiol Biochem 37: 671-678.
45. Clough S J, Bent A F (1998) Floral dip: a simplified method for Agrobacterium-mediated transformation of Arabidopsis thaliana. Plant J 16: 735-743.
46. DalCorso G, Pesaresi P, Masiero S, Aseeva E, Schunemann D, et al. (2008) A complex containing PGRL1 and PGR5 is involved in the switch between linear and cyclic electron flow in Arabidopsis. Cell 132: 273-285.
47. Bolter B, Soll J (2007) Import of plastid precursor proteins into pea chloroplasts. Methods Mol Biol 390: 195-206.
48. Bassi R, dal Belin Peruffo A, Barbato R, Ghisi R (1985) Differences in chlorophyll-protein complexes and composition of polypeptides between thylakoids from bundle sheaths and mesophyll cells in maize. Eur J Biochem 146: 589-595.
49. Pesaresi P, Varotto C, Meurer J, Jahns P, Salamini F, et al. (2001) Knock-out of the plastid ribosomal protein L11 in Arabidopsis: effects on mRNA translation and photosynthesis. Plant J 27: 179-189.

1. A plant cell or plant, wherein the activity of a phosphatase of the light harvesting complex of photosystem II has been reduced or inhibited.

2. The plant cell or plant according to claim 1, wherein said phosphatase has an N-terminal chloroplast transit peptide (cTP), a C-terminal transmembrane domain and a protein phosphatase 2C signature.

3. The plant cell or plant according to claim 1, wherein the activity of said phosphatase of the light harvesting complex of photosystem has been reduced or inhibited by one of the following:
(i) producing a mutant nucleic acid coding for a non-functional version of said phosphatase, through insertion, deletion or mutation of at least one nucleotide in the nucleic acid wildtype coding sequence of said phosphatase, and introducing said mutant nucleic acid into a plant cell,
(ii) producing a mutant nucleic acid coding for a version of said phosphatase having reduced expression, through insertion, deletion, mutation of at least one nucleotide at a position 1-500 nucleotides upstream of the nucleic acid wildtype coding sequence of said phosphatase, and introducing said mutant nucleic acid into a plant cell,
(iii) producing a mutant nucleic acid by placing the nucleic acid coding for said phosphatase under the control of a regulatory sequence that switches off the expression of said nucleic acid coding for said phosphatase, and introducing said mutant nucleic acid into a plant cell,
(iv) producing, within a plant cell, a mutant nucleic acid coding for a non-functional version of said phosphatase or a version of said phosphatase having reduced expression. by exposing said plant cell to a mutagenic substance, or by exposing said plant cell to mutagenic radiation, and identifying the resultant mutant nucleic acid based on the recognition of mismatch hybridizations,
(v) introducing siRNA directed at the nucleic acid coding for said phosphatase, into a plant cell, or
(vi) blocking the activity of said phosphatase within a plant cell at the protein level by exposing said plant cell to a phosphatase inhibitor.

4. The plant cell or plant according to claim 1, wherein said plant cell or plant is selected from monocotyledons, dicotyledons, or eudicotyledons.

5. The plant cell or plant according to claim 4, wherein said plant cell or plant is from a plant useful for human nutrition or animal nutrition, and/or from a flowering plant and/or from a greenhouse plant and/or from a plant useful for the production of biomass.

6. The plant cell or plant according to claim 4, wherein said plant cell or plant is from a species selected from tomato (Lycopersicon esculentum), corn (Zea mays), wheat (Triticum aestivum), Sorghum, rice (Oryza sativa), barley (Hordeum vuigare), cucumber (Cucumis sativus), courgette or zucchini (Cucurbita pepo), squash or gourd or pumpkin (Cucurbita pepo, Cucurbita mixta, Cucurbita maxima, Cucurbita moschata), eggplant or aubergine (Solanum melongena), lettuce (Lactuca sativa), spinach (Spinacia oleracea), cabbage (Brassica oleracea), chicory (Cichorium intybus), corn salad (Valerianella locusta), broccoli (Brassica oleracea), cauliflower (Brassica oleracea), common grape vine (Vitis vinifera), sugar beet (Beta vulgaris), beetroot (Beta vulgaris), thale cress (Arabidopsis thaliana), castor oil plant (Ricinus communis), Western Balsam Poplar (Populus Trichocarpa), Sitka spruce (Picea sitchensis); herb species such as from basil (Ocimum basilicum), thyme (Thymus subsp.), sage (such as Salvia species), chivers (Allium schoenoprasum), parsley (Petroselinum crispum, Petroselinum neapolitanum), berry species, Ribes species, Vaccinium species, Rubus species, potato (Solanum tuberosum), carrot (Daucus carota), leek (Allium ampeloprasum), onion (Allium cepa), Piper species, Capsicum species, melon species, okra (Abehnoschus esculentus), beans, Vigna species, Phaseolus species, asparagus (Asparagus officinalis), artichoke (Cynara cardunculus), celeriac (Apium graveolens), peas (Pisum sativum); Citrus species or Actinidia species, Chinese cabbage (Brassica rapa), rapeseed (Brassica napus), tobacco (Nicotiana tabacum), radish (Raphanus sativus), orange (citrus sinensis), wild radish (Raphanus raphanistrum), Columbine species (Aquilegia sp.), clementine (Citrus clementina), owl's clover species (Triphysaria sp.). safflower (Carthamus tinctorius), mandarin (Citrus reticulata), upland cotton (Gossypium hirsutum), sunflower (Helianthus annuus), soybean (Glycine max), oat species, switehgrass (Panicum virgatum), annual wormwood (Artemisia annua), and nut species.

7. The plant cell or plant according to claim 1, wherein said phosphatase is a phosphatase of the light harvesting complex of photosystem II and has an amino acid sequence which is
a) SEQ ID NO:1,
b) a sequence that is at least 50% identical to SEQ ID NO:1, or
c) a homologue to SEQ ID NO:1 from a plant species other than Arabidopsis thaliana, said homologue having an N-terminal chloroplast transit peptide (cTP), a C-terminal transmembrane domain and a protein phosphatase 2C signature.

8. The plant cell or plant according to claim 7, wherein said homologue has an amino acid sequence selected from SEQ ID NO:2-11 and 23-47.

9. The plant cell or plant according to claim 1, wherein said plant cell or plant has an increased photosynthesis rate or increased production of biomass in comparison with a plant cell or plant, wherein said phosphatase has not been inhibited.

10. A method of increasing the photosynthesis rate and/or the production of biomass in a plant cell or plant, said method comprising:
reducing or inhibiting, in said plant cell or plant, the activity of a phosphatase of the light harvesting complex of photosystem II.

11. The method according to claim 10, wherein said phosphatase has an amino acid sequence which is
a) SEQ ID NO: 1,
b) a sequence that is at least 50% identical to SEQ ID NO: 1, or
c) a homologue to SEQ ID NO: 1 from a plant species other than Arabidopsis thaliana, said homologue having an N-terminal chloroplast transit peptide (cTP), a C-terminal transmembrane domain and a protein phosphatase 2C signature.

12. The method according to claim 11, wherein said homologue has an amino acid sequence selected from SEQ ID NO: 2-11 and 23-47.

13. The method according to claim 10, wherein said reducing or inhibiting the activity of said phosphatase occurs by one of the following:
(i) producing a mutant nucleic acid coding for a non-functional version of said phosphatase, through insertion, deletion or mutation of at least one nucleotide in the nucleic acid wildtype coding sequence of said phosphatase, and introducing said mutant nucleic acid into a plant cell,
(ii) producing a mutant nucleic acid coding for a version of said phosphatase having reduced expression, through insertion, deletion, mutation of at least one nucleotide at a position 1-500 nucleotides upstream of the nucleic acid wildtype coding sequence of said phosphatase, and introducing said mutant nucleic acid into a plant cell,
(iii) producing a mutant nucleic acid by placing the nucleic acid coding for said phosphatase under the control of a regulatory sequence that switches off the expression of said nucleic acid coding for said phosphatase, and introducing said mutant nucleic acid into a plant cell,
(iv) producing, within a plant cell, a mutant nucleic acid coding for a non-functional version of said phosphatase or a version of said phosphatase having reduced expression, by exposing said plant cell to a mutagenic substance, or by exposing said plant cell to mutagenic radiation, and identifying the resultant mutant nucleic acid based on the recognition of mismatch hybridizations,
(v) introducing siRNA directed at the nucleic acid coding for said phosphatase, into a plant cell, or
(vi) blocking the activity of said phosphatase within a plant cell at the protein level by exposing said plant cell to a phosphatase inhibitor.

14. The method according to claim 10, wherein increasing the photosynthesis rate and/or the production of biomass occurs under light exposure of said plant cell or plant to photosynthetic active radiation (PAR) in the range of 50-200 μmol m⁻² s⁻¹.

15. The method according to claim 13, wherein the nucleic acid coding for said phosphatase has a nucleic acid sequence selected from SEQ ID NO: 12-22 and 48-72.

16. The method according to claim 13, wherein the siRNA consists of 15 to 21, or 21 to 50, or 50 to 100, or 100 to 150, or 150 to 200 contiguous nucleotides of SEQ ID NO: 12-22 and 48-72.

17. The method according to claim 13, wherein said mutant nucleic acid is a mutant nucleic acid the expression of which in a plant cell results in at least 80% fewer transcripts of said phosphatase, said mutant nucleic acid having an insertion, deletion or mutation of at least one nucleotide in the nucleic acid wildtype coding sequence of said phosphatase, or said mutant nucleic acid having an insertion, deletion or mutation of at least one nucleotide upstream of the nucleic acid wildtype coding sequence of said phosphatase.

18. The method according to claim 17, wherein said mutant nucleic acid has a nucleic acid sequence selected from SEQ ID NO: 12-22 and 48-72 into which,. or upstream of which at least one nucleotide has been inserted.

19. The method according to claim 10, wherein said plant cell or plant is selected from monocotyledons or dicotyledons or eudicotyledons, useful for human nutrition or animal nutrition, or from a greenhouse plant or from a plant useful for the production of biomass.
